(19) United States
(12) Patent Application Publication
(10) Pub. No.: US 2021/0220481 A1
(43) Pub. Date: Jul. 22, 2021

(54) BIOCOMPATIBLE TOLEROGENIC NANOPARTICLES

(71) Applicants: INSERM (Institut National de la Santé et de la Recherche Médicale), Paris (FR); Assistance Publique - Hopitaux de Paris, Paris (FR); Université Paris 13, Villetaneuse (FR); Université de Paris, Paris (FR); Centre National de la Recherche Scienifique (CNRS), Paris (FR)

(72) Inventors: Peter VAN ENDERT, Paris (FR); Laurence MOTTE, Bobigny (FR); Chloé DUBREIL, Paris Cedex 15 (FR)

(21) Appl. No.: 17/265,554

(22) PCT Filed: Jul. 24, 2019
(86) PCT No.: PCT/EP2019/069869

§ 371 Date: Feb. 3, 2021

(30) Foreign Application Priority Data
Aug. 3, 2018 (EP) 18187336.5

(51) Int. Cl. A61K 47/69; A61K 38/28; A61K 47/60

(52) U.S. Cl. CPC A61K 47/6929; A61K 47/60; A61K 47/6923; A61K 38/28

(57) ABSTRACT

The present invention relates to nanoparticles, methods and compositions which are suitable for the detection and/or follow-up and/or treatment of type 1 diabetes. In particular, it relates to biocompatible tolerogenic nanoparticles comprising at least: (i) a ligand which can bind to an aryl hydrocarbon receptor (AHR) transcription factor; an (ii) a diabetes autoantigen selected from: insulin, preproinsulin, proinsulin, or an immunologically active fragment thereof The inventors have shown that such biocompatible tolerogenic nanoparticles are efficient for the identification of type-1 diabetes. It has also been shown that they can accumulate into the pancreas, and induce temporary or lasting remission of disease in spontaneously diabetic NOD mice. Kits and compositions are further provided.

### FIELD OF THE INVENTION

The present invention relates to nanoparticles, methods and compositions which are suitable for the detection, diagnosis and/or follow-up, and/or treatment of type 1 diabetes.

### BACKGROUND OF THE INVENTION

Chronic autoimmune diseases are the consequence of the recognition by the immune system of self-antigens (autoantigens) as foreign, which can lead to inflammation and destruction of the targeted tissues and organs. Type 1 diabetes (T1D) is one of the most common chronic autoimmune diseases. T1D is characterized by insulin deficiency due to selective destruction of insulin-producing β-cells caused by autoreactive T-cells infiltrating pancreatic Langerhans islets inducing islet inflammation. Beta cell damage can begin months or years before clinical diagnosis, which is generally characterized by hyperglycemia causing polyuria, polydipsia and polyphagia. At clinical onset, more than 70% of the β-cell mass can be destroyed. Consequently, early diagnosis is a major objective in order to avoid, limit or reverse autoimmune aggression, and to create opportunities for strategies enhancing β-cell survival or regeneration.

Antigen (Ag)-specific approaches are appealing because their effects are expected to be limited to cells expressing the chosen antigen, ideally the target organ. However, while treatment with β-cell Ags can prevent disease in the model of the Non-Obese Diabetic (NOD) mouse clinical trials in humans have produced disappointing results. The difficulty of inducing regulatory T cell (Treg) responses in an auto-inflammatory setting at T1D onset likely represents a major obstacle. Consequently, combinatorial approaches may be required for reversal and prevention of T1D

A potential strategy is to associate self-antigens with signals inducing a tolerogenic phenotype in APCs. For example, the 2-(1′H-indole-3′-carbonyl)-thiazole-4-carboxylic acid methylester (ITE), an endogenous non-toxic Aryl hydrocarbon Receptor (AhR) ligand isolated originally from lung, has been reported to induce a tolerogenic phenotype in DCs, promoting the differentiation of CD4+ cells into Treg cells. It was shown that the tolerogenic signal is provided by the activation of AhR.

Recent studies suggest that combining autoantigen and immunomodulator has the potential to produce promising results in various autoimmune diseases.

Co-delivery ensures that both compounds will be delivered at the same time and presented in the same environment to auto-reactive immune cells.

For instance, Yeste et al. (“Tolerogenic nanoparticles inhibit T cell-mediated autoimmunity through SOCS2”; Sci Signal; 2016; 9(433)) and WO2016154362 suggest to administer 60-nm gold nanoparticles loaded with (i) a β-cell antigen derived from insulin and (ii) the tolerogenic AhR ligand 2-(1′H-indole-3′-carbonyl)-thiazole-4-carboxylic acid methyl ester (ITE), in order to suppress the spontaneous development of T1D in non-obese diabetic (NOD) mice. However, as stated by Serra & Santamaria (Eur.J.Immunol.2018.48:751-756) the ability to reverse established type-1 diabetes (otherwise said, to “treat” established type-1 diabetes) was not tested in those documents. Also, their accumulation in the pancreas and pancreatic lymph nodes was not established. In particular, the above-mentioned documents did not provide any clear characterization of the nanoparticle material. Also, applications to in vivo imaging were not discussed.

In order to develop such strategies and to apply them to a wider range of autoimmune diseases, it is important to undertake a thorough physicochemical characterization of the drug vector, aiming to establish quantitative methods optimizing drug loading while maintaining biocompatibility and stability of the delivery vehicle. In the field of nanomedicine, such parameters can be limiting points

Thus, there remains a need to identify novel reagents for the treatment of autoimmune disorders, and in particular of type 1-diabetes.

There also remains a need for biocompatible reagents for the diagnosis of such autoimmune disorders, and in particular of type 1-diabetes.

There also remains a need for reagents which can be used as imaging agents, in particular for magnetic resonance imaging (MRI).

The invention has for purpose to meet the above-mentioned needs.

### SUMMARY OF THE INVENTION

The invention relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is for use for treating type-I diabetes.

The invention also relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is for use for the in vivo diagnosis of type-I diabetes.

The invention also relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is a magnetic nanoparticle.

The invention also relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle has an average size of less than about 50 nm.

The invention also relates to a composition comprising a contrast agent, in combination with a biocompatible tolerogenic nanoparticle, wherein the said nanoparticle comprises at least: (i) a ligand which can bind to an aryl hydrocarbon receptor (AHR) transcription factor; and (ii) a diabetes autoantigen selected from: insulin, preproinsulin, proinsulin, or an immunologically active fragment thereof.

The invention also relates to a kit comprising:
a first container containing a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof;
a second container containing a contrast agent.

The invention also relates to a method for preparing a contrast composition, comprising a step of bringing into contact a contrast agent with a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof.

### BRIEF DESCRIPTION OF THE FIGURES

FIG. 1. Characterization of USPIO-PEG loaded with biomolecules: (A) Cartoon depicting tolerogenic nanoparticles loaded with ITE trapped between PEG chains and fusion protein (proinsulin; Ub: Ubiquitin: streptococcal protein G covalently linked via peptide bond; (B) and (C) TEM images at two magnification of USPIO-PO-PEG NPs; (D) TEM size distribution of USPIO-PEG NPs; (E) hydrodynamic size and zeta potential versus pH; (F) hydrodynamic diameter of USPIO-PEG NPs measured in water, NaCl 0.9% and NaCl 0.9%/Glucose 5% at pH 7.4 and 37° C.; (G) FTIR; (H) magnetization curve at room temperature; (I) ZFC/FC curve; (J) UV spectra of free ITE at various concentrations after basic hydrolysis and of USPIO-PEG-ITE NPs after the same treatment; (K) calibration curve of the 2 characteristic UV absorbance bands (279 and 388 nm) of carboxylate ITE after decomposition by basis hydrolysis;. (L) FTIR spectra of USPIO-PEG, free P3UmPI and of USPIO-PEG after loading with P3UmPI at a initial ratio of P3UmPI/NP equal to 8.

FIG. 2. Quantification of NP internalization by BMDCs. (A) using magnetometry for UPSIO-PEG; USPIO-PEG-ITE, USPIO-PEG-P3UmPI; USPIO-PEG-ITE-P3UmPI (B) Amount of P3umPi (on the left y-axis and appearing as squares) and ITE (on the right y-axis and appearing as triangles) internalized per cell in BMDCs after 24 h incubation and % of internalized NPs. (C) BMDC viability was assessed 24 h after NP incubation using flow cytometry (from left to right for each condition: UPSIO-PEG; USPIO-PEG-ITE; USPIO-PEG-P3UmPI; USPIO-PEG-ITE-P3UmPI. (D) Quantitative PCR analysis of AhRR gene expression in BMDCs treated with vehicle, free ITE or USPIO-PEG-ITE (mean±S.E.M.; n=3 experiments). (E) ImmunoBlot analysis of P3UmPI and USPIO-PEG-P3UmPI.

FIG. 3. Nanoparticle biodistribution after injection. MRI contrast variation showing NP biodistribution after injection in B6 mice (A) and in NOD mice (B). From left to right, histograms correspond respectively to the contrast at 30 min, 1 h, 5 h, 24 h and 72 h after injection.

FIG. 4. NP biodistribution analysis after injection. in B6 (A and C) and NOD mice (B and D) using magnetometry measurement. From left to right, the histograms correspond to the % of injected NPs at 1 h, 5 h, 24 h, 48 h and 72 h after injection. LMF magnetization curve analysis of B6 mouse liver ( [symbol] ) kidneys ( [symbol] ) and NOD mouse liver ( [symbol] ) kidney ( . . . ) inset: magnetic size distribution.

FIG. 5. Characterization of USPIO synthesis. USPIO synthesis (A) and TEM size distribution (B) hydrodynamic size peak and zeta potential vs pH (C) characterization (magnetization curve at room temperature and ZFC/FC curve)

FIG. 6. Thermogravimetric Analysis (TGA) of PEG and USPIO-PEG

FIG. 7. Characterization of USPIO-PEG loaded with ITE. FTIR spectra of (A) USPIO-PEG (upper curve), ITE (medium curve) and USPIO-PEG after loading with R ITE/NP=600 (lower curve). (B) FTIR spectra of USPIO-PEG and ITE mixed in various proportions; inset: linear increase of the C—O band area (normalized with respect to the Fe—O band) with the number of ITE molecules.

FIG. 8. In vitro transverse relaxivity measurements. At 37° C. and 7 T of USPIO-PEG (A) and USPIO-PEG-ITE-P3UmPI (B) measured in 0.3% agar by a 7T MRI.

FIG. 9. Blood NPs biodistribution analysis. After injection in B6 (A) and NOD mice (B) using magnetometry measurement. 1 H, 24 H, 48 H and 72 H after injection.

FIG. 10. ITE drug release evaluation over time using quantitative (UV spectroscopy) assay. NPs dispersed in NaCl 0.9%/Glucose 5%. Corresponding values are defined further in Table 2.

FIG. 11. In vivo evaluation of the effect of an injection of nanoparticles on glycemia in diabetic NOD mice. (A) Protocol of injection consisting of injecting NPs, particles loaded with P3UmPI and/or ITE, twice weekly over four weeks. (B) Percent survival is indicated on the y-axis and time is expressed in days after disease onset on the x-axis. (C) Percent survival over time of mice treated with NPs comprising PEG+ITE+P3UmPI and displaying glycemia below or above 350 mg/dL at start of treatment.

FIG. 12. Profile of splenic and PLN immune cells in mice cured by complete nanoparticle treatment. From left to right: each dot represents the number of given splenic cell population in untreated C57BL/6, prediabetic, diabetic mice and in cured mice treated with nanoparticles of the invention. The following markers are quantified in the y-axis: (A) CD45+ splenocytes (B) splenic T cells (C) splenic CD4+ cells (D) splenic CD4+ Foxp3+ cells (E) splenic CD8+ cells (F) splenic dendritic cells/macrophages (G) splenic B cells. Similar results were obtained with pancreatic lymph node cells.

FIG. 13. Memory phenotype and IFN-γ production by T cells in cured mice. (A,B) The ratio of memory (CD4+CD62L−) to naïve (CD44−CD62L+) cells was determined for splenic CD4+ and CD8+ T cells in control C57BL/6 mice as well as prediabetic, diabetic and cured NOD mice. Panels (C,D) show the percentage of IFN-γ-producing CD4+ and CD8+ T cells in the spleen of the four groups of mice.

### DETAILED DESCRIPTION OF THE INVENTION

The inventors have hypothesized that nanoparticulate contrast agents might accumulate in inflamed pancreatic islets via the Enhanced Permeability and Retention (EPR) effect, inducing changes in tissue contrast.

Herein the inventors report iron oxide nanoparticles (NPs) which were surface-engineered for the co-packaging of the autoantigen proinsulin, a major target of adaptive immunity in Type 1 diabetes (T1D), and ITE, a small drug conditioning a tolerogenic environment. Magnetic resonance imaging (MRI) combined to magnetic quantification were used to investigate NP biokinetics in non-obese diabetic (NOD) mice and control mice in different organs.

According to an exemplified embodiment, ultrasmall superparamagnetic iron-oxide (USPIO) NPs are surface functionalized with phosphonate polyethylene glycol (PEG) molecules with brush conformation and with a carboxyl-terminal function (USPIO-PEG). The carboxylic acid functions were used to covalently bind the T1D autoantigen proinsulin in the form of a fusion protein, referred herein as “P3UmPI”, through carbodiimide coupling. This fusion protein corresponds to a fusion protein containing proinsulin, ubiquitin and tandem streptococcal immunoglobulin-binding domains, as disclosed in Kratzer et al. (J Immunol 184 (2010): 6855-64). The PEG brush contributes to the co-packaging of the tolerogenic and hydrophobic ITE molecules, trapped between PEG chains through hydrophobic interactions.

The inventors now report different NP biodistribution, with enhanced kidney elimination and stronger accumulation in the pancreas for pre-diabetic NOD mice. This has been related to preferential NP accumulation in the pancreatic inflammatory zone and to enhancement of renal elimination by diabetic nephropathy. Accordingly, the inventors report herein a MRI T2 contrast enhancement at 72 h in liver, pancreas and kidneys, which indicates re-circulating NPs. Moreover, on the base of those biodistribution results, different metabolic routes engaged by NPs are identified in pre-diabetic NOD versus control mice. This unexpected result was confirmed by magnetic quantification at different time points as well as by histological evaluation.

In particular, the inventors demonstrate that PEGylated iron oxide NPs, according to the invention, accumulate preferably in the pancreas of NOD mice via the EPR effect, thus enabling the identification of pre-diabetic mice or diabetic mice from non-diabetic controls.

The inventors thus confirm that NPs of the invention can be potential MRI contrast agents for the early diagnosis of T1D. This result also supports that vascular leakage, as a strategy for early diagnosis of T1D, can be used to improve NP bioaccumulation, both for delivery of therapeutic agents and for use as imaging agents, especially to monitor Type-1 diabetes (T1D). Thus, the inventors have designed nanoparticles which are particularly efficient as T2 MRI contrast agents, especially for the follow-up of Type-1 diabetes.

More specifically, the inventors have shown that NPs of the invention shorten T2 relaxation time, thereby reducing signal intensity on T2-weighted images, by increasing transverse relaxivity r2 of the loaded platform. These r₂ values are higher than those of commercial MRI contrasts agents, thus leading to a large negative contrast enhancement which was observed across organs rich in macrophages, i.e. liver, kidneys, spleen but also in pancreas.

Nanoparticles (NPs) of the invention are thus defined as biocompatible tolerogenic nanoparticle comprising at least: (i) a ligand which can bind to an aryl hydrocarbon receptor (AHR) transcription factor; and (ii) a diabetes autoantigen.

Most preferably, the ligand which can bind to an aryl hydrocarbon receptor (AHR) transcription factor is the tolerogenic AhR ligand 2-(1′H-indole-3′-carbonyl)-thiazole-4-carboxylic acid methyl ester (ITE).

Most preferably, the diabetes autoantigen is a polypeptide comprising a sequence selected from the group consisting of preproinsulin or an immunologically active fragment thereof; such as an immunologically active fragment of proinsulin.

Overall, nanoparticles of the invention are particularly relevant for use as diagnostic and/or imaging device; in particular for the preparation of MRI contrast agents. Thus, nanoparticles of the invention preferably possess magnetic, especially superparamagnetic, properties.

Most-preferably, the nanoparticles of the invention are iron-oxide nanoparticles. Advantageously, the use of iron oxide also renders those nanoparticles more biodegradable than other types of nanoparticles, such as gold nanoparticles.

The inventors propose herein that nanoparticles according to the invention, especially ultrasmall superparamagnetic iron-oxide (USPIO) nanoparticles (NPs), display excellent biocompatibility and physical properties, due notably to (i) finite size effects, such as the high surface-to-volume ratio, (ii) unique features for drug loading and drug delivery in various vascular pathologies including notably enhanced permeability and retention (EPR) effect, and (iii) convenient surface reactivity, allowing NP surface functionalization with therapeutics and/or targeting molecules.

Without wishing to be bound by the theory, and considering the combined effects of each layer of the glomerular capillary walls, it is also expected that small NP with a small hydrodynamic diameter (HD) can pass through the glomerular capillary wall easily, while larger ones would not be filtrated. This is particularly sought after, as kidney filtration is a desirable pathway for NP clearance because potential health hazards resulting from long-term accumulation and decomposition of NPs in the body can be minimized.

Moreover, the inventors report herein that such nanoparticles can accumulate in the pancreas, and induce temporary or lasting remission of disease in spontaneously diabetic NOD mice; thus identifying a novel strategy for the treatment of established type-1 disease.

According to a first embodiment, the invention relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is for use for treating type-I diabetes.

According to a second embodiment, the invention relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is for use for the in vivo diagnosis of type-I diabetes.

According to a third embodiment, the invention relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle is a magnetic nanoparticle.

According to a fourth embodiment, the invention relates to a biocompatible tolerogenic nanoparticle comprising at least: (i) a ligand which can bind to an aryl hydrocarbon receptor (AHR) transcription factor; and (ii) a diabetes autoantigen selected from: insulin, preproinsulin, proinsulin, or an immunologically active fragment thereof;
wherein the said nanoparticle has an overall size of less than about 50 nm.

According to a fifth embodiment, the invention relates to a composition comprising a contrast agent, in combination with a biocompatible tolerogenic nanoparticle, wherein the said nanoparticle comprises at least: (i) a ligand which can bind to an aryl hydrocarbon receptor (AHR) transcription factor; and (ii) a diabetes autoantigen selected from: insulin, preproinsulin, proinsulin, or an immunologically active fragment thereof.

According to a sixth embodiment, the invention relates to a kit comprising:
a first container containing a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof;
a second container containing a contrast agent.

According to a seventh embodiment, the invention relates to a method for preparing a contrast composition, comprising a step of bringing into contact a contrast agent with a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof.

### DEFINITIONS

As used herein, the term “type-1 diabetes”, or “insulin-dependent diabetes”, refers to any form of diabetes which can be characterized by a deficient, or insufficient, insulin production, as defined by the World Health Organization (see Diabetes Fact sheet No 312). For instance, this term may encompass patients resulting from the pancreas's failure to produce enough insulin, whether the cause is known or unknown. This term may also encompass patients still having a normal fasting glycaemia but developing a type-1 diabetes, for instance because they harbour functionally impaired and/or a reduced mass of insulin-producing beta cells in the pancreatic islets. This term may also encompass patients having an impaired fasting glycaemia thus having a clinically manifest type-1 diabetes, as discussed above.

On the other hand, the population of patients characterized by the occurrence of “type-1 diabetes” does not encompass “type-2 diabetes” or “gestational diabetes”, or intermediate conditions referred as “impaired glucose tolerance (IGT)” or “impaired fasting glycaemia (IFG)” which are not associated with type-1 diabetes.

As used herein, “treating” means any manner in which one or more of the symptoms of a disease or disorder are ameliorated or otherwise beneficially altered. As used herein, amelioration of the symptoms of a particular disorder refers to any lessening of the symptoms, whether permanent or temporary, lasting or transient, that can be attributed to or associated with treatment by the compositions and methods of the present invention. Accordingly, the expression “treating” may include “reversing partially or totally the effect” of a given condition, or even “curing” when permanent reversal is considered. It flows from the above that, in the context of a “treatment of type-1 diabetes”, this term shall be interpreted to encompass the treatment of a subject/patient, or of a group of subjects/patients, which actually have, or are presumed to have, type-1 diabetes. However it does necessarily flow that the targeted patients are all at the same stage of the disease. Accordingly, the present invention is not restricted to the treatment of patients or groups of patients which are at a late stage of the disease, but it may also concern patients or groups of patients at an early stage of the disease.

As used herein, “treating a type-1 diabetes” may thus comprise “reducing, arresting, reversing partially or totally the loss of insulin producing beta cells of the pancreatic islets, whether directly or indirectly”. It may also include the symptomatic treatment of type-1 diabetes, including “normalizing and/or reducing glycemia” in a type-1 disease patient.

For groups of subjects who do not have, or are not presumed to have, type-1 diabetes, the term “preventing” is preferred. As used herein, “preventing” encompasses “reducing the likelihood of occurrence” and “reducing the likelihood of re-occurrence”.

The terms “effective amount” and “effective to treat,” as used herein, refer to an amount or a concentration of one or more of the compositions described herein utilized for a period of time (including acute or chronic administration and periodic or continuous administration) that is effective within the context of its administration for causing an intended effect or physiological outcome.

As used herein, the term “subject” or “patient” may encompass an animal, human or non-human, rodent or non-rodent. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

As used herein, a “diagnosis” may also encompass the “follow-up” of a given patient or population of patients over time. When the patient was not previously diagnosed, this term may also encompass the “detection” of type-1 diabetes.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, «a plurality of» may thus include «two» or «two or more».

As used herein, «comprising» may include «consisting of».

As used herein, an “immunologically active fragment” generally refers to a fragment of a given antigen (e.g. preproinsulin or proinsulin) having at least five (5) consecutive amino acids from the said antigen. Thus, this definition may encompass fragments having at least 5, 6, 7, 8, 9, 10, 11, 12, 13, 14, 15, 16, 17, 18, 19, 20, 21, 22, 23, 24, 25, 26, 26, 27, 28, 29, or 30 consecutive aminoacids from the said antigen.

As used herein, a “contrast agent”, or “contrast medium”or “contrast composition”, refers to a composition suitable for highlighting specific organs, blood vessels, or tissues.

As used herein, an “imaging agent” refers mostly to any contrast agent, which is susceptible to be used to increase the contrast of structures or fluids within a biological sample or body of a subject, especially in the context of medical imaging (e.g. the follow-up of a subject or a group of subjects presumed to have diabetes, which may include discriminating between subjects having diabetes (especially type-1 diabetes) and subjects not having diabetes). In particular, an imaging agent can be a Magnetic Resonance Imaging (MRI) contrast agent. Hence, a “MRI contrast agent” is a particular type of imaging agent which is suitable for increasing the contrast of structures or fluids within a biological sample or body of a subject.

A Magnetic Resonance Imaging (MRI) contrast agent can be classified by, e.g., chemical composition, administration route, magnetic properties, effect on the image, metal center's presence and nature, or biodistribution or applications. MRI contrast agents classified by biodistribution can include: extracellular fluid agents (also known as intravenous contrast agents); blood pool agents (also known as intravascular contrast agents); organ specific agents (i.e., gastrointestinal contrast agents and hepatobiliary contrast agents); active targeting/cell labeling agents (i.e. tumor-specific agents); responsive (also known as smart or bioactivated) agents; and pH-sensitive agents.

Such MRI contrast agents shorten the relaxation times of nuclei within body tissues following oral or intravenous administration. A MRI contrast agent of the present disclosure can be a T₁, T₂, and T₂* contrast agent that can be administered as described herein.

As used herein, the term “imaging composition” refers to a composition comprising at least one imaging agent. This term may thus encompass a composition comprising a contrast agent prepared by reacting or combining parahydrogen-enriched hydrogen with a hydrogenatable magnetic resonance imaging agent precursor or substrate, and/or a nanoparticle of the invention.

As used herein, the term “biocompatible” is meant to refer to compounds (e.g. nanoparticles) which do not cause a significant adverse reaction in a living animal when used in pharmaceutically relevant amounts.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration. The use of such media and agents for pharmaceutically active substances are known. Except insofar as any conventional media or agent is incompatible with the active compound, such media can be used in the compositions of the invention.

As used herein, the term “tolerogenic” is meant to refer to compounds (e.g. nanoparticles) which are able to induce immune tolerance where there is pathological or undesirable activation of the normal immune response.

As used herein, the terms “magnetic” and “superparamagnetic” is meant to refer to magnetic and superparamagnetic behavior at room temperature.

As used herein, “Aryl hydrocarbon receptor (AhR)” refers to a transcription factor that upon activation by its ligand 2-(1 [symbol] H-indole-3 [symbol] -carbonyl)-thiazole-4-carboxylic acid methyl ester (ITE) or other ligands induces tolerogenic dendritic cells (DCs) that promote the generation of regulatory T cells. AhR is a basic helix-loop-helix/PAS domain containing ligand-activated transcription factor that, once activated, can bind to specific DNA motif sequences (called xenobiotic response elements or XREs) and initiate transcription, as described in Nebert et al (J Biol Chem 279(23):23847-23850, 2004).

Hence, as used herein, a “ligand which can bind to an aryl hydrocarbon receptor (AHR) transcription factor» refers to a ligand (for instance, a naturally-occurring, recombinant or synthetic polypeptide) which can bind to the Aryl hydrocarbon Receptor, in a manner susceptible to activate the Aryl hydrocarbon Receptor and generate a tolerogenic signal in Antigen-Presenting Cells (APC), such as dendritic cells (DC). Particular examples of such ligands are described hereafter.

As used herein, the term “nanoparticles” is meant to refer to particles having an average size (such as a diameter, for spherical or nearly spherical nanoparticles) of 100 nanometres (nm) in size or less. The “diameter” is typically defined as the “crystalline diameter” or as the “hydrodynamic diameter”. The crystalline size (or “diameter” if applicable) of a population of nanoparticles can be determined herein by transmission electron microscopy whereas the hydrodynamic size related to surface functionalization is measured by dynamic laser light scattering (DLS), in a physiological medium, for example NaCl 0.9% , NaCl 0.9%/Glucose 5%, or other buffer media at a physiological pH, used for biological evaluation as well as in vitro and in vivo experiments, as described in the Material & Methods section.

As a general reference, the average hydrodynamic size (or “diameter”) is most preferably determined in a physiological medium corresponding to NaCl 0.9%/Glucose 5% at pH 7.4 and 37° C. Even though spherical nanoparticles are particularly considered in the context of the invention, it will be understood herein that the term “nanoparticle” is not meant to refer exclusively to one type of shape. Accordingly, this term may also encompass other shapes, selected from: spherical nanoparticles, rod-shaped nanoparticles, vesicle-shaped nanoparticles, and S-shaped worm-like particles as described in Hinde et al. (“Pair correlation microscopy reveals the role of nanoparticle shape in intracellular transport and site of drug release”; Nature nanotechnology; 2016) as well as other morphologies such as nanoflower, raspberry, and core-shell nanoparticles.

Nanoparticles of the invention can include targeting moieties, in addition to (i) a ligand which can bind to an aryl hydrocarbon receptor (AHR) transcription factor, and (ii) a diabetes autoantigen.

As used herein, the terms “targeting moiety” and “targeting agent” are used interchangeably and are intended to mean any agent, such as a functional group, that serves to target or direct the nanoparticle to a particular location or association (e.g., a specific binding event). Thus, for example, a targeting moiety may be used to target a molecule to a specific target protein or enzyme, or to a particular cellular location, or to a particular cell type, to selectively enhance accumulation of the nanoparticle. Suitable targeting moieties include, but are not limited to, polypeptides, nucleic acids, carbohydrates, lipids, hormones including proteinaceous and steroid hormones, growth factors, receptor ligands, antigens and antibodies, and the like. For example, as is more fully outlined below, the nanoparticles of the invention may include a targeting moiety to target the nanoparticles (including biologically active agents associated with the nanoparticles) to a specific cell type, such as liver, spleen, pancreas or kidney cell type.

As used herein, the term “lipid” includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

As used herein, the term “linked to”, such as in “a ligand linked to the nanoparticles” may refer either to a covalent link or to a non-covalent link. In a non-limitative manner, such non-covalent interactions may occur due to electrostatic interactions, Van der Walls forces, π-effects, and hydrophobic effects. Alternatively, covalent-interactions occur as a consequence of the formation of a covalent bond, such as the coupling of a ligand which can bind to an aryl hydrocarbon receptor (AHR) transcription factor, and a functional (reactive) chemical group at the surface of the nanoparticle.

### Nanoparticles of the Invention

As previously stated, nanoparticles (NPs) of the invention are defined as biocompatible tolerogenic nanoparticle comprising at least: (i) a ligand which can bind to an aryl hydrocarbon receptor (AHR) transcription factor; and (ii) a diabetes autoantigen.

Most preferably, the ligand which can bind to an aryl hydrocarbon receptor (AHR) transcription factor is the tolerogenic AhR ligand 2-(1′H-indole-3′-carbonyl)-thiazole-4-carboxylic acid methyl ester (ITE).

Most preferably, the diabetes autoantigen is a polypeptide comprising a sequence selected from the group consisting of preproinsulin or an immunologically active fragment thereof; such as an immunologically active fragment of proinsulin.

This ligand and diabetes autoantigen may be either comprised (e.g. encapsulated) within the nanoparticle or attached (linked covalently or non-covalently) in a matter suitable for release into and/or contact with the surrounding medium (i.e. at the surface of the nanoparticle).

Most preferably, the nanoparticles are attached (linked covalently or non-covalently) to the AhR ligands and the diabetes autoantigen described herein (e.g. via functional groups). When applicable, such functional groups may be born by a polymer such as, but not limited to, polyethylene glycol (PEG).

A nanoparticle of the invention may be linked covalently or non-covalently to at least: (i) a ligand which can bind to an aryl hydrocarbon receptor (AHR) transcription factor; and (ii) a diabetes autoantigen.

According to an exemplified embodiment, the nanoparticle may be:
covalently bound to a ligand which can bind to an aryl hydrocarbon receptor (AHR) transcription factor; and
non-covalently bound to a diabetes autoantigen.

According to some other embodiments, the nanoparticle may be:
covalently bound to a ligand which can bind to an aryl hydrocarbon receptor (AHR) transcription factor; and
covalently bound to a diabetes autoantigen.

According to some embodiments, the nanoparticle may be:
covalently bound to a ligand which can bind to an aryl hydrocarbon receptor (AHR) transcription factor; and
non-covalently bound to a diabetes autoantigen.

According to some other embodiments, the nanoparticle may be:
non-covalently bound to a ligand which can bind to an aryl hydrocarbon receptor (AHR) transcription factor; and
covalently bound to a diabetes autoantigen.

According to some other embodiments, the nanoparticle may be:
non-covalently bound to a ligand which can bind to an aryl hydrocarbon receptor (AHR) transcription factor; and
non-covalently bound to a diabetes autoantigen.

Most preferably, nanoparticles of the invention are magnetic nanoparticles; and especially nanoparticles having superparamagnetic properties.

Advantageously, nanoparticles of the invention are metal-oxide nanoparticles.

Most preferably, nanoparticles of the invention are ultrasmall superparamagnetic iron-oxide (USPIO) nanoparticles.

Other particular embodiments of the nanoparticles (NPs) which are relevant in the context of the invention are described hereafter.

The nanoparticles which are particularly considered, and useful in the methods and compositions described herein, are made of materials that are (i) biocompatible e.g. do not cause a significant adverse reaction in a living animal when used in pharmaceutically relevant amounts; (ii) feature functional groups to which the binding moiety can be covalently attached, (iii) exhibit low non-specific binding of interactive moieties to the nanoparticle, and (iv) are stable in solution, e.g., the nanoparticles do not precipitate.

The nanoparticles can be monodisperse (a single crystal of a material, e.g., a metal, per nanoparticle) or polydisperse (a plurality of crystals, e.g., 2, 3, or 4, per nanoparticle).

A number of biocompatible nanoparticles are known in the art, e.g., organic or inorganic nanoparticles. Liposomes, dendrimers, carbon nanomaterials and polymeric micelles are examples of organic nanoparticles. Quantum dots can also be used. Inorganic nanoparticles include metallic nanoparticle, e.g., Au, Ni, Pt and TiO₂ nanoparticles. Magnetic nanoparticles can also be used, e.g., spherical nanocrystals of 10-20 nm with a Fe2+ and/or Fe3+ core surrounded by dextran or PEG molecules.

Metal-oxide nanoparticles, such as iron-oxide nanoparticles, are especially considered. In some embodiments, colloidal gold nanoparticles can be used, e.g., as described in Qian et al. (Nat. Biotechnol. 26(1):83-90 (2008)); U.S. Pat. Nos. 7,060,121; 7,232,474; and US2008/0166706. Suitable nanoparticles, and methods for constructing and using multifunctional nanoparticles, are discussed in e.g., Sanvicens and Marco (Trends Biotech., 26(8): 425-433 (2008)).

Spherical nanoparticles are particularly considered. Alternatively, non-spherical nanoparticles are also considered, as described hereafter.

According to some other embodiments, the nanoparticles of the invention may also include micelle-shaped nanoparticles, vesicle-shaped nanoparticles, rod-shaped nanoparticles, and worm-shaped nanoparticles as described for instance in Hinde et al. (“Pair correlation microscopy reveals the role of nanoparticle shape in intracellular transport and site of drug release”; Nature nanotechnology; 2016).

In some embodiments, the nanoparticles have an average size (or “diameter”) of about 1-100 nm, e.g., about 25-75 nm, e.g., about 40-60 nm, or about 50-60 nm. The polymer component in some embodiments can be in the form of a coating, e.g., about 5 to 20 nm thick or more. According to a most preferred embodiment, the nanoparticles have an average size (or «diameter») of less than about 50 nm. Thus, a nanoparticle (or population thereof) having an average size (or «diameter») of less than about 100 nm; which includes nanoparticles (or populations thereof) having an average size (or «diameter») of less than about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, and 3 nm.

In an illustrative manner, nanoparticles having an average size of about 3 nm are reported in Richard et al. (Nanomedicine (Lond) 2016. DOI 10.2217/nnm-2016-0177).

Methods for the functionalization of nanoparticles are known in the Art. Accordingly, reference is made to Perrier et al. («Methods for the Functionalisation of Nanoparticles: New Insights and Perspectives»; 2010; Chem. Eur. J. 2010, 16, 11516-11529). In a non-limitative manner, such functional groups may comprise or consist of one or more functional groups selected from: alkyl, alkenyl, alkynyl, phenyl, halo, fluoro, chloro, bromo, iodo, hydroxyl, carbonyl, aldehyde, haloformyl, carbonate ester, carboxylate, ester, methoxy, hydroperoxy, peroxy, ether, hemiacetal, hemiketal, acetal, ketal, orthoester, methylenedioxy, orthocarbonate ester, carboxalide, amine, imine, imide, azide, azo(diimide), cyanate, isocyanate, nitrate, nitrile, isonitrile, nitrosooxy, nitro, nitroso, oxime, pyridyl, sulfhydryl, sulfide, disulfide, sulfinyl, sulfonyl, sulfino, sulfothiocyanate, isothiocyanate, thiol, carbonothioyl, phosphino, phosphono, phosphate, borono, boronate, borino, and borinate functional groups.

In some embodiments, nanoparticles of the invention can be associated with a polymer that includes functional groups.

When applicable, this also serves to keep the metal oxides dispersed from each other. The polymer can be a synthetic polymer, such as, but not limited to, polyethylene glycol (PEG) or silane, natural polymers, or derivatives of either synthetic or natural polymers or a combination of these.

Useful polymers are hydrophilic. In some embodiments, the polymer “coating” is not a continuous film (i.e. a continuous film around a magnetic metal oxide), but is a “mesh” or “cloud” of extended polymer chains attached to and surrounding the metal oxide. The polymer can comprise polysaccharides and derivatives, including dextran, pullanan, carboxydextran, carboxmethyl dextran, and/or reduced carboxymethyl dextran. When applicable, the metal oxide can be a collection of one or more crystals that contact each other, or that are individually entrapped or surrounded by the polymer.

Thus, nanoparticles of the invention may also consist of porous nanoparticles such as metal organic framework (MOF) nanoparticles, which can be functionalized and used as effective carriers for drug delivery. Metal-organic frameworks, also referred herein as “porous coordination polymers (PCPs)” can be generally defined as coordination polymers of hybrid inorganic-organic framework containing metal ions and organic ligands coordinated to the metal ions. These materials are organized into one-, two- or three-dimensional frameworks where the metal clusters are bound together by spacer ligands in a periodic manner. These materials have a crystalline structure, are most often porous and are used in many industrial applications such as the storage of gas, the adsorption of liquids, the separation of liquids or gases, catalysis, and more recently medical applications.

According to preferred embodiments, the biocompatible tolerogenic nanoparticle of the invention is a magnetic nanoparticle; in particular a nanoparticle having superparamagnetic properties.

According to exemplified embodiments, the biocompatible tolerogenic nanoparticle of the invention is a iron oxide nanoparticle.

According to a preferred embodiment, the biocompatible tolerogenic nanoparticle of the invention comprises a ligand which can bind to an aryl hydrocarbon receptor (AHR) transcription factor is 2-(1′H-indole-3′-carbonyl)-thiazole-4-carboxylic acid methyl ester (ITE).

According to a preferred embodiment, the biocompatible tolerogenic nanoparticle of the invention comprises a sequence selected from the group consisting of insulin, preproinsulin, proinsulin or an immunologically active fragment thereof.

According to a preferred embodiment, the biocompatible tolerogenic nanoparticle of the invention has an average size (or «diameter») of less than about 50 nm.

According to a preferred embodiment, the biocompatible tolerogenic nanoparticle is functionalized with phosphonate polyethylene glycol (PEG) molecules.

According to said preferred embodiment, the biocompatible tolerogenic nanoparticle has, even more preferably, an average density of PEG molecules at the surface of the nanoparticle ranging from 0.1 to 5 PEG per nm², such as from 0.5 to 2 PEG per nm².

According to a preferred embodiment, the biocompatible tolerogenic nanoparticle the nanoparticle is linked to the ligand which can bind to an AHR transcription factor with an average density of ligand at the surface of the nanoparticle from 0.5 to 4 ligands per nm²; the said ligand being preferably ITE.

### Diabetes Autoantigen

Most preferably, the diabetes autoantigen is a polypeptide comprising a sequence selected from the group consisting of preproinsulin, proinsulin, or an immunologically active fragment thereof.

As known in the Art, preproinsulin corresponds to proinsulin with a signal peptide attached to its N-terminus.

According to one embodiment, the diabetes autoantigen is a polypeptide comprising a sequence selected from the group consisting of insulin, preproinsulin, proinsulin, or an immunologically active fragment thereof.

Thus, the diabetes autoantigen may be a polypeptide comprising at least five consecutive amino acids from insulin, preproinsulin, or proinsulin; and most preferably at least five consecutive amino acids from proinsulin.

For reference, a polypeptide sequence of human insulin is reported in the UniProtKB datase (reference P01308) along some of its variants.

According to some embodiments, the diabetes autoantigen may be in the form of a fusion protein. A “fusion protein” refers to a protein artificially created from at least two amino-acid sequences of different origins, which are fused either directly (generally by a peptide bond) or via a peptide linker.

The diabetes autoantigen may be in the form of a fusion protein, characterized in that it comprises an IgG binding moiety and, as a cargo moiety, a polypeptide comprising a sequence selected from the group consisting of insulin, preproinsulin, proinsulin, or an immunologically active fragment thereof.

The IgG binding moiety may consist of at least two IgG binding domains. In particular, the IgG binding moiety may consist of at least two IgG binding domains of streptococcal protein G placed in tandem arrangement.

Thus, according to some embodiments, the diabetes autoantigen may be in the form of a fusion protein characterised in that it comprises:
an IgG binding moiety consisting of at least two IgG binding domains of streptococcal protein G placed in tandem arrangement;
as a cargo moiety, a polypeptide comprising a sequence selected from the group consisting of insulin, preproinsulin, proinsulin, or an immunologically active fragment thereof.

Especially, the cargo moiety may comprise an ubiquitin domain fused to the N-terminal or C-terminal end of the polypeptide. However, in the case wherein enhanced degradation of the polypeptide of interest (i.e. insulin, preproinsulin, proinsulin or an immunologically fragment thereof) by ubiquitin fusion is desired, the ubiquitin domain should be fused directly to the N-terminal end of the polypeptide of interest.

Peptide linkers may be employed to separate two or more of the different components of a fusion protein of the invention. In particular, peptide linkers will advantageously be inserted between the IgG binding domains in the IgG binding moiety, and between the IgG binding moiety and the cargo moiety. Peptide linkers are classically used in fusion proteins in order to ensure their correct folding into secondary and tertiary structures. They are generally from 2 to about 50 amino acids in length, and can have any sequence, provided that it does not form a secondary structure that would interfere with domain folding of the fusion protein.

According to one exemplary embodiment, the diabetes autoantigen is a fusion protein which is coupled to tandem immunoglobulin-binding domains from streptococcal protein G and ubiquitin. One example of such fusion protein may be as described in WO2008035217.

Regarding diabetes autoantigens specifically, reference is also made to Kratzer et al. (J Immunol 184 (2010): 6855-64) which teaches a P3UmPI fusion protein comprising a proinsulin antigen of murine origin, which can advantageously be substituted by a human insulin, preproinsulin or proinsulin polypeptide sequence.

According to some embodiments, the ratio of diabetes autoantigen vs Nanoparticules (autoantigen/NP) ranges from 1 to about 400; which includes from 1 to about 50; which includes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50. According to particular embodiments, the ratio (autoantigen/NP) ranges from 1 to 20, which includes from 3 to 15.

According to some embodiments, the ratio of proinsulin autoantigen vs Nanoparticules (proinsulin/NP) ranges from 1 to about 400; which includes from 1 to about 50; which includes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50. According to particular embodiments, the ratio (proinsulin/NP) ranges from 1 to 20, which includes from 3 to 15.

### AHR Transcription Factor Ligands

Examples of ligands which can bind to an aryl hydrocarbon receptor (AHR) transcription factor, and which are suitable in the context of the invention, include the high affinity AHR ligand 2,3,7,8-tetrachlorodibenzo-p-dioxin (TCDD), tryptamine (TA), and/or 2-(1′H-indole-3′-carbonyl)-thiazole-4-carboxylic acid methyl ester (ITE). Other potential AhR transcription factor ligands are described in Denison and Nagy (Ann. Rev. Pharmacol. Toxicol., 43:309-34, 2003), all of which are incorporated herein in their entirety. Other such molecules include planar, hydrophobic HAHs (such as the polyhalogenated dibenzo-pdioxins, dibenzofurans, and biphenyls) and PAHs (such as 3-methylcholanthrene, benzo(a)pyrene, benzanthracenes, and benzo flavones), and related compounds. (Denison and Nagy, 2003, supra).

Nagy et al., Toxicol. Sci. 65:200-10 (2002), described a high-throughput screen useful for identifying and confirming other ligands. See also Nagy et al. (Biochem. 41:861-68 (2002). In some embodiments, those ligands useful in the present invention are those that bind competitively with TCDD, TA, and/or ITE.

Most preferably, the ligand which can bind to the aryl hydrocarbon receptor (AHR) transcription factor, is ITE. AhR ligands can also include structural analogs of 2-(1′H-indole-3′-carbonyl)-thiazole-4-carboxylic acid methyl ester (ITE), which are described in WO2016154362.

Accordingly, in some embodiments, the AhR ligands can include compounds having the following formula:

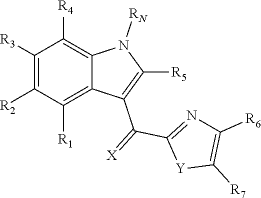

wherein X and Y, independently, can be either O (oxygen) or S (sulfur);
R_{N} can be selected from hydrogen, halo cyano formyl alkyl haloalkyl alkenyl alkynyl, alkanoyl, haloalkanoyl, or a nitrogen protective group;
R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy;
R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy;
or R₆ and R₇, independently, can be:

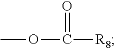

wherein R₈ can be selected from hydrogen, halo, cyano, alkyl, haloalkyl, alkenyl, or alkynyl;
or R₆ and R₇, independently, can be:

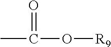

wherein R₉ can be selected from hydrogen, halo, alkyl, haloalkyl, alkenyl, or alkynyl;
or R₆ and R₇, independently, can be:

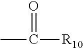

wherein R₁₀ can be selected from hydrogen, halo, hydroxy, thiol, cyano, alkyl, haloalkyl, alkenyl, alkynyl, amino, or nitro;
or R₆ and R₇, independently, can also be:

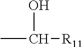

wherein R₁₁ can be selected from hydrogen, halo, alkyl, haloalkyl, alkenyl, or alkynyl.

In some embodiments, the structure of the 2-(1′H-indole-3′-carbonyl)-thiazole-4-carboxylic acid methyl ester analog is represented by one of the following formulas:

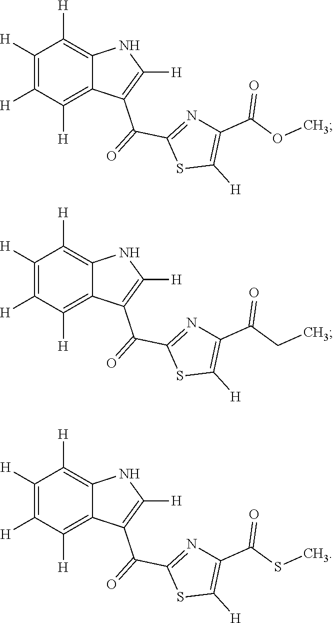 [SMILES: COC(=O)c1csc(C(=O)c2c[nH]c3ccccc23)n1 ; CCC(=O)c1csc(C(=O)c2c[nH]c3ccccc23)n1 ; CSC(=O)c1csc(C(=O)c2c[nH]c3ccccc23)n1]

Other such molecules include polycyclic aromatic hydrocarbons exemplified by 3-methylchoranthrene (3-MC); halogenated aromatic hydrocarbons typified by 2,3,7,8-tetrachlorodibenzo-p-dioxin (TCDD); planar, hydrophobic HAHs (such as the polyhalogenated dibenzo-p-dioxins and dibenzofurans (e.g., 6-methyl-1,3,8-trichlorodibenzofuran or 6-MCDF), 8-methyl-1,3,6-trichlorodibenzofuran (8-MCDF)), and biphenyls) and polycyclic aromatic hydrocarbons (PAHs) (such as 3-methylcholanthrene, benzo(a)pyrene, benzanthracenes, and benzo flavones), and related compounds).

Naturally-occurring AHR ligands can also be used, e.g., tryptophan catabolites such as indole-3-acetaldehyde (IAAlD), indole-3-aldehyde (IAlD), indole-3-acetic acid (IAA), tryptamine (TrA), kynurenine, kynurenic acid, xanthurenic acid, 5-hydroxytryptophan, serotonin; and Cinnabarinic Acid (Lowe et al., PLoS ONE 9(2): e87877; Zelante et al., Immunity 39, 372-385, Aug. 22, 2013; Nguyen et al., Front Immunol. 2014 Oct. 29; 5:551); biliverdin or bilirubin (Quintana and Sherr, Pharmacol Rev 65:1148-1161, October 2013); prostaglandins (PGF3a, PGG2, PGH1, PGB3, PGD3, and PGH2); leukotrienes, (6-trans-LTB 4, 6-trans-12-epi-LTB); dihydroxyeicosatriaenoic acids (4,5(S),6(S)-DiHETE, 5(S),6(R)-DiHETE); hydroxyeicosatrienoic acid (12(R)-HETE) and lipoxin A4 (Quintana and Sherr, Pharmacol Rev 65:1148-1161, October 2013).

In some embodiments, the AHR ligand is a flavone or derivative thereof, e.g., 3,4-dimethoxyflavone, 3′-methoxy-4′-nitroflavone, 4′,5,7-Trihydroxyflavone (apigenin) or 1-Methyl-N-[2-methyl-4-[2-(2-methylphenyl)diazenyl]phenyl-1H-pyrazole-5-carboxamide; resveratrol (trans-3,5,4′-Trihydroxystilbene) or a derivative thereof; epigallocatechin or epigallocatechingallate.

In some embodiments, the AHR ligand is one of the 1,2-dihydro-4-hydroxy-2-oxo-quinoline-3-carboxanilides, their thieno-pyridone analogs, and prodrugs thereof, e.g., having the structure:

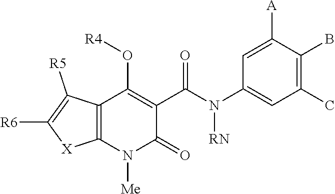

wherein
A, B and C are independently chosen from the group comprising H, Me, Et, iso-Pr, tert-Bu, OMe, OEt, O-iso-Pr, SMe, S(O)Me, S(O)2 e, CF3, OCF3, F, CI, Br, I, and CN, or A and B represents OCH₂O and C is H;
RN is chosen from the group comprising H, C(O)H, C(O)Me, C(O)Et, C(O)Pr, C(O)CH(Me)₂, C(O)C(Me)₃, C(O)Ph, C(O)CH2Ph, CO₂H, CO₂Me, CO₂Et, CO₂CH₂Ph,
C(O)NHMe, C(O)NMe₂, C(O)NHEt, C(O)NEt₂, C(O)NHPh, C(O)NHCH2Ph, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(O)Me, S(O)₂Me, S(O)₂NMe₂, CF₃, OCF₃, F, CI, OH, CO₂H, CO₂Me, CO₂Et, C(O)NH₂, C(O)NMe₂, NH₂, NH₃, NMe₂, NMe₃+, NHC(O)Me, NC(═NH)NH₂, OS(O)₂OH, S(O)₂OH, OP(O)(OH)₂, and P(O)(OH)₂;
R₄ is RN, or when RN is H, then R₄ is chosen from the group comprising H, P(O) (OH)₂, P(O) (OMe)₂, P(O) (OEt)₂, P(O) (OPh)₂, P(O) (OCH2Ph)₂, S(O)₂OH, S(O)₂NH₂, S(O)₂NMe₂, C(O)H, C(O)Me, C(O)Et, C(O)Pr, C(O)CH(Me)₂, C(O)C(Me)₃, C(O)Ph, C(O)CH₂Ph, CO₂H, CO₂Me, CO₂Et, CO₂CH₂Ph, C(O)NHMe, C(O)NMe₂, C(O)NHEt, C(O)NEt₂, C(O)NHPh, C(O)NHCH₂Ph, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(O)Me, S(O)2Me, S(O)2NMe₂, CF3, OCF₃, F, CI, OH, CO₂H, CO₂Me, CO₂Et, C(O)NH₂, C(O) Me₂, NH₂, NH₃+, Me₂, NMe₃+, NHC(O)Me, NC(═NH)NH₂, OS(O)₂OH, S(O)₂OH, OP(0) (OH)₂, and P(O)(OH)₂;
R₅ and R₆ are independently chosen from the group comprising H, Me, Et, iso-Pr, tert-Bu, OMe, OEt, O-iso-Pr, SMe, S(O)Me, S(O)₂Me, CF₃, OCF₃, F, Cl, Br, I, and CN, or R₅ and R₆ represents OCH₂O; and X is —CH═CH—, or S, or pharmaceutically acceptable salts of the compounds thereof.

In some embodiments, the AHR ligand is laquinimod (a 5-Cl, N-Et carboxanilide derivative) or a salt thereof (see, e.g., US20140128430).

In some embodiments, the AHR ligand is characterized by the following general formula:

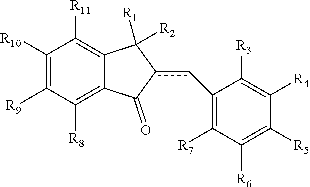

wherein
(i) R₁ and R₂ independently of each other are hydrogen or a C₁ to C₁₂ alkyl,
(ii) R₃ to R₁₁ independently from each other are hydrogen, a C₁ to C₁₂ alkyl, hydroxyl or a C₁ to C₁₂ alkoxy, and
(iii) the broken line represents either a double bond or two hydrogens.

In some embodiments, the AhR ligand has one of the following formulae:

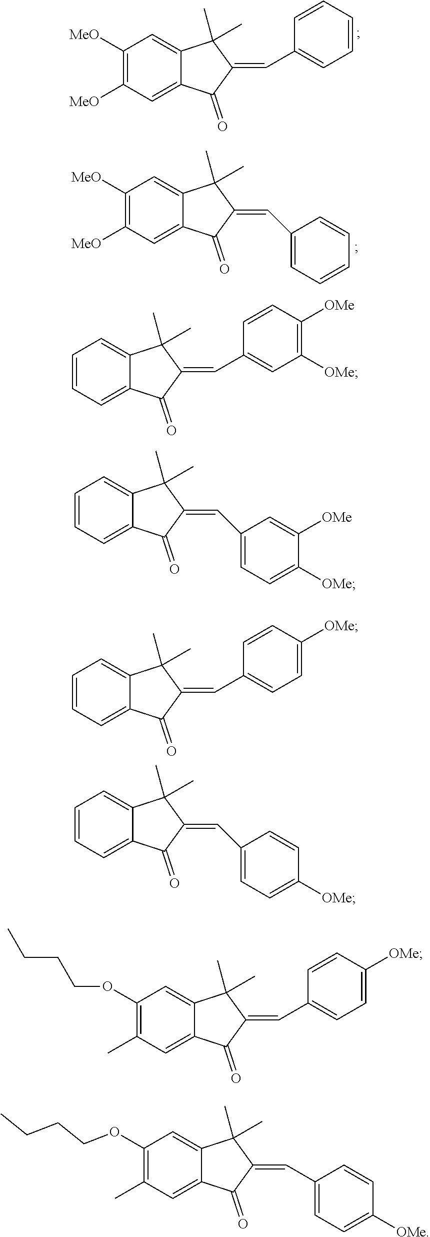

In some embodiments, the AhR ligand has a general formula of:

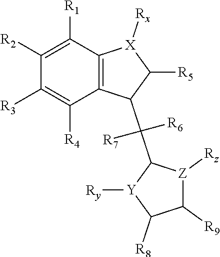

wherein:
R₁, R₂, R₃ and R₄ can be independently selected from the group consisting of hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl and carbonyloxy.
R₅ can be selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
R₆ and R₇ together can be ═O.
Alternatively, R₆ can be selected from the group consisting of hydrogen, halo, cyano, formyl, alkyl, haloalkyl, alkenyl, alkynyl, alkanoyl and haloalkanoyl, and R₇ is independently selected from the group consisting of hydrogen, halo, hydroxy, thiol, cyano, formyl, alkyl, halo alkyl, alkenyl, alkynyl, amino, nitro, alkoxy, haloalkoxy, thioalkoxy, alkanoyl, haloalkanoyl and carbonyloxy.
Alternatively, R₇ can be selected from the group consisting of hydrogen, halo, cyano, formyl, alkyl, haloalkyl, alkenyl, alkynyl, alkanoyl and haloalkanoyl, and R₆ is independently selected from the group consisting of hydrogen, halo, hydroxy, thiol, cyano, formyl, alkyl, halo alkyl, alkenyl, alkynyl, amino, nitro, alkoxy, haloalkoxy, thioalkoxy, alkanoyl, haloalkano 1 and carbonyloxy.
R₈ and R₉, independently, can be

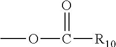

and R₁₀ is selected from the group consisting of hydrogen, halo, cyano, alkyl, haloalkl, alkenyl and alkynyl.
Alternatively, R₈ and R₉, independently, can be

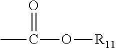

and R₁₁ is selected from the group consisting of hydrogen, halo, alkyl, haloalkyl, alkenyl and alkynyl.
Alternatively, R₈ and R₉, independently, can be

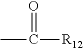

and R₁₂ is selected from the group consisting of hydrogen, halo, hydroxy, thiol, cyano, alkyl, haloalkyl, alkenyl, alkynyl, amino and nitro.
Alternatively, R₈ and R₉, independently, can be

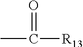

and R₁₃ is selected from the group consisting of hydrogen, halo, alkyl, halo alkyl, alkenyl and alkynyl.
Alternatively, R₈ and R₉, independently, can be selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
X can be oxygen or sulfur, and Rx is nothing. Alternatively, X can be nitrogen, and Rx is selected from the group consisting of hydrogen, halo, formyl, alkyl, haloalkyl, alkenyl, alkynyl, alkanoyl, haloalkanoyl and a nitrogen protective group. Alternatively, X can be carbon, and Rx is selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
Y can be oxygen or sulfur, and Ry is nothing. Alternatively, Y can be nitrogen, and Ry is selected from the group consisting of hydrogen, halo, formyl, alkyl, haloalkyl, alkenyl, alkynyl, alkanoyl, haloalkanoyl and a nitrogen protective group. Alternatively, Y can be carbon, and Ry is selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
Z can be oxygen or sulfur, and Rz is nothing. Alternatively, Z is nitrogen, and Rz is selected from the group consisting of hydrogen, halo, formyl, alkyl, haloalkyl, alkenyl, alkynyl, alkanoyl, haloalkanoyl and a nitrogen protective group.
Alternatively, Z can be carbon, and Rz is selected from hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
Other AHR ligands include stilbene derivatives and flavone derivatives of formula I and formula II, respectively:

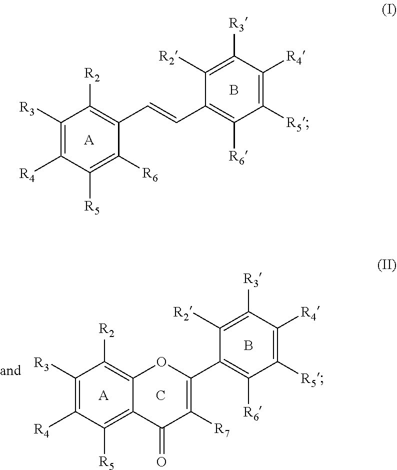

wherein R₂, R₃, R₄, R₅, R₆, R₇ and R₂′ R₃′, R₄′, R₅′, R₆′ are identical or different (including all symmetrical derivatives) and represent H, OH, R (where R represents substituted or unsubstituted, saturated or unsaturated, linear or branched aliphatic groups containing one to thirty carbon atoms), Ac (where Ac represents substituted or unsubstituted, saturated or unsaturated, cyclic compounds, including alicyclic and heterocyclic, preferably containing three to eight atoms), Ar (where Ar represents substituted or unsubstituted, aromatic or heteroaromatic groups preferably containing five or six atoms), Cr (where Cr represents substituted or unsubstituted fused Ac and/or Ar groups, including Spiro compounds and norbornane systems, preferably containing two to five fused rings), OR, X (where X represents an halogen atom), CX₃, CHX₂, CH₂X, glucoside, galactoside, mannoside derivates, sulfate and glucuronide conjugates. Optical and geometrical isomeric derivatives of stilbene and flavone compounds are included.

Among the compounds encompassed by the general formulas I and II are apigenin, tangeritin (4′,5,6,7,8-pentamethoxyflavone), diosmin (5-Hydroxy-2-(3-hydroxy-4-methoxyphenyl)-7-[(2S,3R,4S,5S,6R)-3,4,5-trihyd-roxy-6-[[(2R,3R,4R,5R,6S)-3,4,5-trihydroxy-6-methyloxan-2-yl]oxymethyl]oxa-n-2-yl]oxychromen-4-one), flavoxate (2-(1-piperidyl)ethyl 3-methyl-4-oxo-2-phenyl-chromene-8-carboxylate), piceatannol (3,4,3′,5′-tetrahydroxystilbene), oxyresveratrol (2,3′,4,5′-tetrahydroxystilbene), 4,4′-dihydroxystilbene.

In a non-limitative manner, such ligands may thus be selected from a list including: 2,3,7,8-Tetrachlorodibenzo-p-dioxin; 3,4,3′5-Pentachlorobiphenyl; 2,3,7,8-Tetrachlorodibenzofuran; 3-Methylcholanthrene; Benzo(a)pyrene; β-Naphthoflavone; YH439; Thiabendazole; Omeprazole; SKF71739; (1S, 2R)-(-)-cis-1-Amino-2-indanol; 5-Methyl-2-phenylindole; 2(Methylmercapto)aniline; 1-Methyl-1-phenylhydrazine; 1,5-Diaminonaphtalene; Guanabenz; SRN-P2:109,NH₂; 2-4-Amino-3-methylphenyl)benzothiazole; Indolo[3,2-b]carnazole; 3,3′-Diindolylmethane; Indirubin; Indigo; Trypthantrin; Malassezin; Tryptamine; Indoleacetic acid; L-Kynurenine; Indole Pyruvic Acid; 6-Formylindolo[3,2-b]carbazole; 6,12-Diformylindolo[3,2-b]carbazole; Dibenzoylmethane; Lipoxin A4; Bilirubin; Tangeritin; Tamarixetin; Prostanglandin G2; Diosmin; Curcumin; Canthaxantin; 7-Ketocholesterol.

### Compositions and Kits

The invention relates to a composition comprising nanoparticles of the invention. Another aspect of the invention concerns a pharmaceutical composition comprising nanoparticles of the invention and a pharmaceutically acceptable carrier.

The invention also relates to a composition comprising a contrast agent, in combination with a biocompatible tolerogenic nanoparticle, wherein the said nanoparticle comprises at least:
(i) a ligand which can bind to an aryl hydrocarbon receptor (AHR) transcription factor;
(ii) a diabetes autoantigen selected from: insulin, preproinsulin, proinsulin, or an immunologically active fragment thereof.

An effective amount (e.g. therapeutically effective amount) of nanoparticles of the invention can be administered by standard methods, for example, by one or more routes of administration, e.g., by one or more of the routes of administration known to the skilled in the Art, including orally, topically, mucosally, intravenously or intramuscularly.

An effective amount of the nanoparticles described herein can be incorporated into pharmaceutical compositions suitable for administration to a subject, e.g., a human. Such compositions typically include the composition and a pharmaceutically acceptable carrier. Supplementary active compounds can also be incorporated into the compositions, e.g., an inhibitor of degradation of the ligand.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can be included in a container, pack, or dispenser together with instructions for administration. In one aspect, the pharmaceutical compositions can be included as a part of a kit (e.g. for use in the methods described herein).

Thus, the kit may comprises:
a first container containing a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof; and
a second container containing a contrast agent.

In some embodiments the kit comprises one or more doses of a composition described herein. The composition, shape, and type of dosage form for the induction regimen and maintenance regimen may vary depending on a subjects requirements. For example, dosage form may be a parenteral dosage form, an oral dosage form, a delayed or controlled release dosage form, a topical, and a mucosal dosage form, including any combination thereof.

In a particular embodiment, a kit can contain one or more of the following in a package or container: (1) one or more doses of a composition described herein; (2) one or more pharmaceutically acceptable adjuvants or excipients (e.g., a pharmaceutically acceptable salt, solvate, hydrate, stereoisomer, and clathrate); (3) one or more vehicles for administration of the dose; (5) instructions for administration. Embodiments in which two or more, including all, of the components (1)-(5), are found in the same container can also be used. When a kit is supplied, the different components of the compositions included can be packaged in separate containers and admixed immediately before use. Such packaging of the components separately can permit long term storage without loosing the active components' functions. When more than one bioactive agent is included in a particular kit, the bioactive agents may be (1) packaged separately and admixed separately with appropriate (similar of different, but compatible) adjuvants or excipients immediately before use, (2) packaged together and admixed together immediately before use, or (3) packaged separately and admixed together.

### Methods & Uses

As previously stated, nanoparticles of the invention can advantageously be used as a medicament, for the treatment for treating type-I diabetes and for in vivo imaging. In particular nanoparticles of the invention having magnetic properties are particularly useful as MRI imaging agent, and/or in combination with other imaging agents.

Thus, the invention relates to a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof; wherein the said nanoparticule is for use for treating type-I diabetes.

The invention also relates to a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof; wherein the said nanoparticle is for use for the in vivo diagnosis of type-I diabetes.

In one aspect, the present invention thus provides a method for MRI imaging, the method comprising.
administering a contrast agent comprising or consisting of a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof by a patient,
acquiring at least a part of a reference MRI image after applying a first external stimulus to said contrast agent,
acquiring at least a part of a contrast enhanced MRI image after applying a second external stimulus to said contrast agent, and
determining effect, e.g. presence, of the contrast agent from a comparison, e.g. difference, between the contrast enhanced MRI image and the reference image.

The invention also relates to a method for preparing a contrast composition, comprising a step of bringing into contact a contrast agent with a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof.

The invention also relates to a method for preparing a contrast composition, comprising a step of bringing into contact a biocompatible tolerogenic nanoparticle with (i) a ligand which can bind to an aryl hydrocarbon receptor (AHR) transcription factor, and (ii) a diabetes autoantigen selected from: insulin, preproinsulin, proinsulin, or an immunologically active fragment thereof.

In particular, the invention relates to a method for preparing a contrast composition, comprising a step of bringing into contact a biocompatible tolerogenic nanoparticle having an average size of less than about 50 nm with (i) a ligand which can bind to an aryl hydrocarbon receptor (AHR) transcription factor, and (ii) a diabetes autoantigen selected from: insulin, preproinsulin, proinsulin, or an immunologically active fragment thereof.

The invention also relates to the use of a biocompatible tolerogenic nanoparticle comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof; for the preparation of a contrast composition

In the context of in vivo imaging, the invention relates to a method for the in vivo imaging (e.g. magnetic resonance imaging), detection, diagnosis and/or the follow-up of patients having diabetes, especially type-1 diabetes, comprising a step of detecting nanoparticles in patient susceptible to have diabetes; wherein the nanoparticles comprise at least: (i) a ligand which can bind to an aryl hydrocarbon receptor (AHR) transcription factor, and (ii) a diabetes autoantigen selected from: insulin, preproinsulin, proinsulin, or an immunologically active fragment thereof.

Accordingly, the invention also relates to a method for the in vivo imaging (e.g. magnetic resonance imaging) and/or the detection, diagnosis and/or follow-up of patients having diabetes, especially type-1 diabetes, comprising a step of:

a) administering nanoparticles comprising at least: (i) a ligand which can bind to an aryl hydrocarbon receptor (AHR) transcription factor, and (ii) a diabetes autoantigen selected from: insulin, preproinsulin, proinsulin, or an immunologically active fragment thereof; and

b) detecting the nanoparticles in the patient.

According to alternative embodiments, the nanoparticles of the invention can be used in vitro or ex vivo (e.g. in an isolated sample, such as a tissue, or a cell). Thus, the invention also relates to an in vitro method for the characterisation of an isolated sample, comprising the steps of:

a) providing an isolated sample;

b) bringing into contact the isolated sample with a biocompatible tolerogenic nanoparticle comprising at least (i) a ligand which can bind to an aryl hydrocarbon receptor (AHR) transcription factor, and (ii) a diabetes autoantigen selected from: insulin, preproinsulin, proinsulin, or an immunologically active fragment thereof; and

c) detecting the biocompatible tolerogenic nanoparticle in the isolated sample, thereby characterizing the isolated sample.

### EXAMPLES

### A. Materials & Methods

Materials: Reagents for particle synthesis were from Sigma-Aldrich (Saint Louis, Mo., USA); Phosphonate-poly(ethylene glycol) PO-PEG-COOH (SP-1P-10-002, MW 2500 g.mol⁻¹) was purchased from Specific Polymers (Specific polymers, Castries, France). The 2-(1H-Indol-3-ylcarbonyl)-4-thiazolecarboxylic acid methyl ester (ITE) was purchased from Tocris bioscience (Bristol, United Kingdom). 1-(3-Dimethylaminopropyl)-3-ethylcarbodiimide hydrochloride (EDC) was purchased from Alfa Aesar (Karlsruhe, Germany).

Fusion protein expression and purification: The fusion protein P₃UmPI is expressed in BL21DE3 pET16b bacteria. Bacteria are pre-cultured at 37° C. in 20 mL LB broth purchased from Sigma-Aldrich (Saint Louis, Mo., USA). 5 mL of the preculture is cultured 4 hours in 500 mL LB broth, ampicillin (50 μg/mL). Protein expression is induced during 4 hours by Isopropyl-β-D-thiogalactoside from Sigma-Aldrich (Saint Louis, Mo., USA). Bacteria are then pelleted by centrifugation (10 min, 5000 g, 4° C.). The pellet is lysed 30 min on ice in a lysis buffer (Tris 50 mM, NaCl 50mM, TCEP 1 mM, EDTA 0.5 mM, glycerol 5%, pH8), lysozyme 0.2 mg/mL and DNase I 0.1 mg/mL. The mix is sonicated 10 times with 1 min off/on pulse. Triton 1% is added for 15 min and after centrifugation (20,000×g, 1H, 4° C.) the supernatant is passed over a rabbit IgG-Sepharose column. Protein is eluted with a CHAPS1%/CAPS 20 mM buffer. Then the protein is dialyzed overnight (MWCO 8000 kDa) in PBS, glycerol 10%. Finally, the protein is passed through columns for removal of detergent (Pierce™ Detergent Removal Spin Column; Thermo fisher Scientific, Waltham, Mass.) and endotoxin (Endotoxin Removal Spin Column; Thermo fisher). Concentration is then measured with fluorescent assay on Qubit (Thermo fisher).

USPIO-PO-PEG-COOH NP synthesis and surface functionalization: Non-coated NPs were synthesized by the reaction of Iron (III) acetyl acetonate (1.1 mmol) with 10 ml of benzylalcohol at 250° C. during 30 min under microwave irradiation on a Monowave 300 from Anton Paar (Anton-Paar, GmbH, Graz, Austria). The resulting suspension was separated using a neodymium magnet, and the precipitate was washed sequentially with dichloromethane followed by sodium hydroxide solution 1 mol·l⁻¹ and finally ethanol 90% (three times for each wash buffer). The solid was re-dispersed in pH=2 water using an HCl solution at 10⁻¹ mol·l⁻¹ and washed three times by ultracentrifugation (Amicon 30 kDa, Merck Millipore). To coat the NPs with PO-PEG-COOH, both compounds were mixed at pH=2 with an equivalent mass (PO-PEG-COOH/NP) ratio of 10. Then the excess of PO-PEG-COOH coating molecules is removed using ultrafiltration (Amicon 30 kDa, Merck Millipore) and USPIO-PO-PEG-COOH NPs were dispersed in water (Invitrogen™ UltraPure™ DNase/RNase-Free Distilled Water pH=7), Sodium chloride solution 0.9% in water (BioXtra), or NaCl 0.9% (BioXtra)/Glucose (Sigma) 5% and the pH was adjusted at 7.4 using NaOH (10⁻¹ mol·l⁻¹) solutions.

Coupling onto USPIO-PO-PEG-COOH NPs: To load ITE on NPs, ITE in DMSO was added to USPIO-PO-PEG-COOH NPs in water at room temperature with a ratio R_{ITE/NP}=600. After 2 hours under mixing, NPs are washed by ultracentrifugation three times for 15 min (Amicon 30 kDa, Merck Millipore). The coupling of P₃UmPI (37.8 kDa) onto USPIO-PO-PEG-COOH NPs was performed in coupling buffer (PF127 3 g/L, H₃PO₄ 0.5 μmol.L⁻¹, pH=6) in a two-step procedure (activation and conjugation) at 37° C. First, the carboxylic acid functions at the outer surface of the NPs were activated using 1-ethyl-3-(3-dimethylaminopropyl) carbodiimide (EDC, n_{EDC}=5n_{COOH}) at pH=6 during 10 min. The second step was the linkage of the amine function of the protein with the activated carboxylic acid functions on the NPs. The protein is added at pH=6 to the ferrofluid during 1 h at 37° C. The procedure was carried out with the ratio R=n_{P3UmPI}/n_{NP}=8. The NPs are washed by ultracentrifugation three times for 15 min (Amicon 100 kDa, Merck Millipore). The NPs were re-dispersed in water at physiological pH for various physicochemical characterizations.

Physico-chemical characterization: The iron concentration was determined by a colorimetric assay as described in previous work [48] A Vibrating Sample Magnetometer (VSM Quantum Design, Versalab) was used for magnetic characterization. VSM measures the magnetization by cycling the applied field from −30 to +30 kOe with a step rate of 100 Oe.s⁻¹. Measurements were performed on USPIO solutions at [Fe]=12.5 mM (corresponding to [NP]=0.8 μM and [Fe₂O₃]=1 g·l⁻¹) at 300 K. The ZFC curve was obtained by first cooling the system in zero field from 270 K to 50 K. Next, an external magnetic field of 100 Oe was applied, and subsequently the magnetization was recorded with a gradual increase in temperature. The FC curve was measured by decreasing the temperature in the same applied field.

The hydrodynamic size and zeta potential of the NPs ([Fe]=0.25 mM) were investigated by dynamic laser light scattering (DLS), using a Nano-ZS (Red Badge) ZEN 3600 device (Malvern Instruments, Malvern, UK). The stability in physiological medium (NaCl 0.9% and NaCl 0.9%/Glucose 5%, [Fe]=0.25 mM) was studied by measuring over time the evolution of the hydrodynamic size.

UV-Vis spectra were recorded on a Varian Cary 50 Scan UV-vis spectrophotometer. TEM images were obtained using a FEI Tecnai 12 (Philips), and samples were prepared by depositing a drop of NP suspension on carbon-coated copper grids placed on a filter paper. The median diameter is deduced from TEM data measurements, simulating the diameter distribution with a log-normal function, according to the methodology described in de Montferrand et al. (“Size-Dependent Nonlinear Weak-Field Magnetic Behavior of Maghemite Nanoparticles”; Small; 2012; 8(12), 1945-56). The grafting of the PO-PEG-COOH to the surface of the NPs, the ITE loading and the coupling of protein P3UmPI was studied by Fourier transform infra-red (FTIR) analysis.

The FTIR spectra were recorded as thin films on KBr pellets on a Thermo Scientific Nicolet 380 FTIR. Quantification of PO-PEG-COOH coating and grafting per particle was evaluated by thermogravimetric analysis (TGA) using a LabsSys evo TG-DTA-DSC 16000 device from Setaram Instrumentation.

The average number of ITE per NP was evaluated using infrared and UV-Visible spectroscopies. For the infrared spectroscopy method, infrared spectra in KBr pellets of various proportions of ITE mixed with a constant amount of USPIO-PO-PEG-COOH NPs were recorded. Then, the normalized 1735 cm⁻¹ band was used for the establishment of a calibration curve and the average number ITE per nanoparticle was deduced from this curve. For the UV-Visible spectroscopy method: ITE saponification was performed by adding 2 mL NaOH 1 mol.L⁻¹ to ITE or ITE-loaded NP for 2 h (200 μL of a NP [Fe]=3.5 μM). In the latter case, the NPs were isolated from supernatant using magnetic decantation. The resulting carboxylate ion (carboxylate ITE) was water soluble and characterized by two UV bands at 279 and 388 nm. A calibration curve was established after basic hydrolysis of ITE alone and the average number ITE per nanoparticle was deduced from this curve.

The ITE saponification was characterized with NMR experiments. 1H NMR spectra (400 MHz, 258C), were recorded in D20 on a Bruker Avance 400 spectrometer and chemical shifts are reported in parts per million (ppm) on the δ scale.

The coupling efficiency of the fusion protein P₃UmPI conjugation was investigated qualitatively using the o-phthalaldehyde (OPA) method. 50 μL of the sample was diluted in 50 μL of NaOH 2 mol.L⁻¹ and left overnight at 60° C. NPs were separated from supernatant using magnetic decantation. 900 μL of OPA reagent was added to the supernatant and fluorescence measurement at 450 nm was recorded on a SpectroFluorimeter Spex FluoroMax (HORIBA Jobin-Yvon, France with a Hamamatsu 98 photomultiplier). The average number of protein per nanoparticle was deduced from a calibration curve.

Mouse bone marrow dendritic cells: BMDCs were prepared from progenitor cells isolated from the femurs and tibias of female mice between 8 and 10 wk of age, as previously described in Inaba et al. (“Generation of large numbers of dendritic cells from mouse bone marrow cultures supplemented with granulocyte/macrophage colony-stimulating factor”; J Exp Med 1992, 176 (6), 1693-702). Bone marrow cells were plated on 15 cm petri dishes cell culture for a final volume of 20 mL per plate in Iscove's Modified Dulbecco's Medium (IMDM) containing 10% FCS, penicillin/streptomycin 100 μg/ml, L-Glutamine 2 mmol.L⁻¹ and β-mercapthoethanol 50 μmol.L⁻¹, and supplemented with 10 ng/ml murine GM-CSF (R&D Systems) for 8 days. Half of the medium was replaced on day 4 of culture.

NP internalization by cells: BMDCs (14.10⁶ cells/well) were incubated with different batches of NPs diluted in complete IMDM containing 10% FCS, penicillin/streptomycin 100 μg/ml, L-Glutamine 2 mM and β-mercapthoethanol 50 μmol.L⁻¹, for different durations: 4, 6, 12, 24 and 48 h. Then the medium was removed and the cells were washed with culture medium, followed by a 2 h wash-out period (in culture medium alone). The saturation magnetization of the pellet was measured using VSM. The magnetic moment thus recorded (in emu) can directly be converted into grams of iron thanks to the magnetization at saturation of the NP (expressed in emu/g of iron) and consequently the average number of NP taken up by cells. Indeed, the saturate magnetization (Ms) is directly proportional to the amount of nanoparticles since the normalized cellular Ms magnetization curve is remarkably similar to the curve of the nanoparticles in aqueous dispersion, as reported in Mazuel et al. (“Massive Intracellular Biodegradation of Iron Oxide Nanoparticles Evidenced Magnetically at Single-Endosome and Tissue Levels”; ACS Nano; 2016; 10(8); 7627-7638).

NP toxicity: BMDCs were incubated 24 hrs with different batches of NPs at different concentrations [NP]: 0.3 nmol.L⁻¹, 1 nmol.L⁻¹, 3 nmol.L⁻¹, 9 nmol.L⁻¹, 27 nmol.L⁻¹, 81 nmol.L⁻¹. Single cell suspensions were prepared from BMDCs and stained for 30 min at 4° C. Staining buffer was PBS containing 2% FCS, 0.5% EDTA and 0.1% sodium azide. Surface staining was performed with the following mAbs: anti-CD11b (eBioscience, clone M1/70), -CD11c (eBioscience, clone N418) for 30 min. Cell viability was evaluated by flow cytometry (Becton Dickinson Fortessa flow cytometer) after 7-amino-actinomycin D (7-AAD,BD Pharmingen) staining.

ITE biological activity. On day 7 of BMDC culture, cells were incubated 24 h with 14 μmol·L⁻¹ ITE in 0.2% DMSO and equivalent amount of ITE as USPIO-PEG-ITE NPs in water. On day 8, total RNA was prepared by the RNAspin mini kit (GE Healthcare). cDNA was obtained using the RevertAid RT Reverse Transcription Kit (Thermo Scientific) with 1 μg of total RNA. Quantitative PCR (qPCR) was performed with the SYBR Green method using Takyon ROX qPCR SYBR MasterMix blue dTTP (Eurogentec).

Western blot analysis of USPIO-PEG-P3UmPI: USPIO-PEG-ITE-P3UmPI was analyzed by Western blot followed by immunostaining. P₃UmPI and unloaded NPs were used as controls. Electrophoresis was performed using NuPAGE® Novex 4-12% Bis-Tris Gel in MES buffer (Invitrogen). After separation, the proteins were transferred to polyvinylidene difluoride (PVDF) membranes with iBlot® 2 Gel transfer device (Life technology) according to the manufacturer's instructions). Non-specific binding to the PVDF membrane was saturated by exposure to 1% fish skin gelatin in PBS with 0.1% Tween for 1 h before the membranes were incubated 1 h at RT with an insulin B Antibody (clone C-12; Santa cruz), followed by goat-anti-mouse IgG IR800 (Advansta) for 1 h at RT. The Odyssey® Imaging Li-Cor System was used to visualize the immunoreactive bands.

In vivo experiments: All in vivo experiments were conducted in strict accordance with the recommendations of the European Community (86/609/EEC) and the French legislation (decree no. 87/848) for use and care of laboratory animals. NOD and B6 mice were used, bred and housed in specific pathogen-free conditions. The protocol for type 1 diabetes treatment and contrast agent injection was approved by Comité d'éthique pour l'expérimentation animale de Paris Descartes under ID 16-057.

For mechanistic studies, MRI and histological studies, 3 B6 mice/time points aged 10 and 12 weeks were used as control. 3 Female NOD mice/time points were used as pre-diabetic mice between 10 and 12 weeks of age. NPs were administrated intravenously (i.v.) as a bolus of 200 μmol_{Fe}/kg diluted in 200 μL of vehicle (NaCl 0.9%/Glucose 5%).

MRI studies: MRI acquisitions were performed on a 7 T small animal MR scanner (Pharmascan, Bruker Bio-Spin GmbH, Rheinstetten, Germany). Relaxometry measurement: in order to generate T2 weighted images at various concentration of NPs ([Fe]=0.010 to 0.700 mmol·L⁻¹) at 37° C. in agarose gel, and thus calculate T2 maps, a multi-slice-multi-echo sequence (TE=11, 33, 55, 77, 99 ms; 16 echos; echo spacing=11 ms and TR=2500 ms) with a slice thickness of 1 mm was used to generate high-resolution coronal images (Matrix: 192×192, Pixel size: 0.234×0.234 mm). By graphing changes in relaxation rate R2 (R2=1/T2) at different concentration, the slope represents the relaxivity r2.

In vivo MRI: a dedicated whole-body mouse coil was used for the image acquisitions. A TurboRARE-T2 (RAREmethod) sequence (TE=9 ms; TR=3,694.281 ms; NEX=4 and RARE factor=8) was used to generate T2 weighted images (Matrix: 256×256) with a slice thickness of 1 mm 30 minutes after i.v. USPIO-PEG-ITE-P3UmPI injection at the dose of 200 μmol_{Fe}/kg diluted in 200 μL of vehicle (NaCl 0.9%/Glucose 5%). Imaging was performed before injection and at different time points after injection (1 h, 5 h, 24 h and 72 h). The contrast variation C was calculated as C=(It−Iₜ₌₀)/Iₜ₌₀ where Iₜ₌₀ is the MRI signal intensity before NP injection and Iₜ the MRI signal intensity at the time t after injection.

Quantification of NP uptake in organs by VSM: Pancreas, lung, liver, spleen, kidneys and PLN were collected 1 h, 5 h, 24 h, 48 h and 72 h after intravenous injection of USPIO-PEG-ITE-P3UMPI (200 μmol_{Fe}/kg) or vehicle (NaCl 0.9%/Glucose 5%) in NOD and B6 mice. Collected organs were frozen in liquid nitrogen, lyophilized and ground to a powder using a mortar and pestle. Sample magnetism was analyzed by a VSM (VSM Quantum Design, Versalab) at room temperature. Amount of iron per organ was calculated from the NP magnetization at saturation (expressed in emu/g of iron).

Histological studies. Pancreas, lung, liver, spleen, kidneys and PLN were collected 1 h, 5 h, 24 h, 48 h and 72 h after intravenous injection of USPIO-PEG-ITE-P3UMPI (200 μmol_{Fe}/kg) or vehicle (NaCl 0.9%/Glucose 5%) in NOD and B6 mice. Organs were embedded in Tissue-Tek OCT Compound (Tissue-Tek, PA, USA), frozen in liquid nitrogen and kept at −80° C. For tissue analysis, embedded slices had a transversal cross-section thickness of 10 μm. The thick cross-sections were stained with hematoxylin/eosin for topographical examination and with Prussian Blue for iron localization. Digital-slide were acquired and analyzed with a NanoZoomer (Hammatsu, Japan).

Evaluation of ITE loading by FTIR spectroscopy. After mixing USPIO-PEG nanoplatforms with various amounts of free ITE. ITE (pink curve, FIG. SI 3 A) presented three sharp bands at 1745 cm⁻¹ (ester C=0), at 1580 cm⁻¹ (ketone) and 1205 cm⁻¹ (ester C—O). Comparing the FTIR spectra of USPIO-PEG (blue curve) and free ITE (pink curve), the overlapping band at 1745cm −1 could be used for quantification. Indeed, a linear increase of the C═O band area, normalized with the Fe—O vibration band (from 500 to 750 cm⁻¹), was obtained by increasing the amount of free ITE added to the NPs (FIG. SI 3 B and inset). The spectrum of USPIO-PEG-ITE NPs (FIG. SI 3A, green curve) is similar to those of pure ITE and of the USPIO-PEG nanoplatform. From the calibration curve an average of 250 ITE per NP was deduced. However, this amount is within the limiting range of FTIR resolution due to the low area of the 1745cm⁻¹ band, suggesting insufficient sensitivity of the FTIR approach.

### B. Results

### Example 1: USPIO-PEG NP Synthesis and Physico-Chemical Characterization

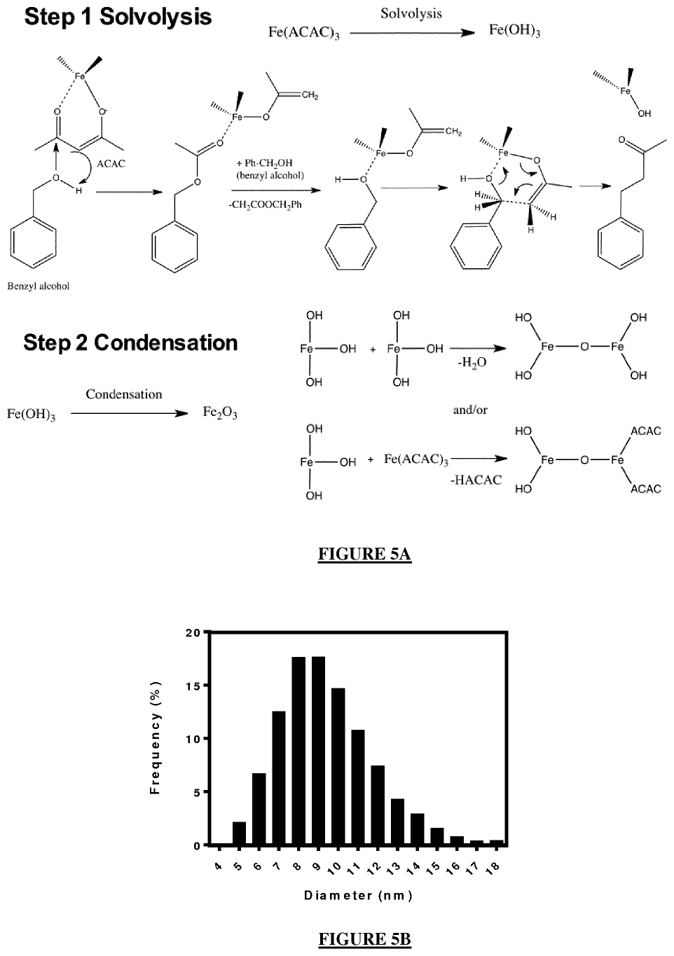

USPIO NPs with an average diameter of 9 nm were synthesized using microwave non aqueous sol-gel synthesis in a two-step process (FIG. 5), following a procedure described in Richard et al. (Nanomedicine (Lond) 2016. DOI 10.2217/nnm-2016-0177). Consistent with their average size, the NPs show superparamagnetic behavior (FIG. 5). The NPs were surface passivated with phosphonate polyethylene glycol (PEG) (n=44; Mw=2100 g mol⁻¹) bearing COOH as terminal functionality.

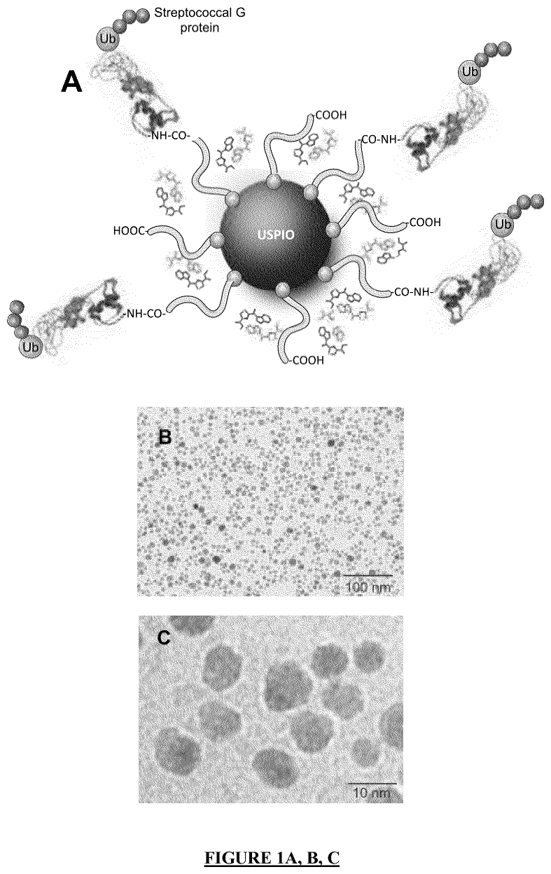

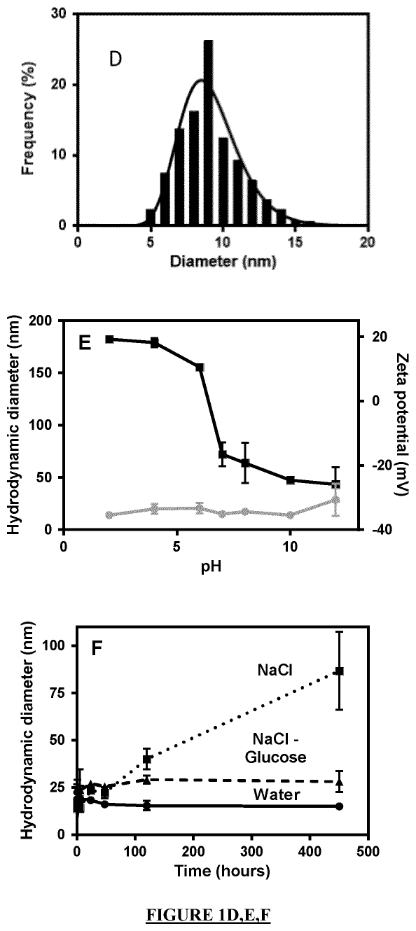

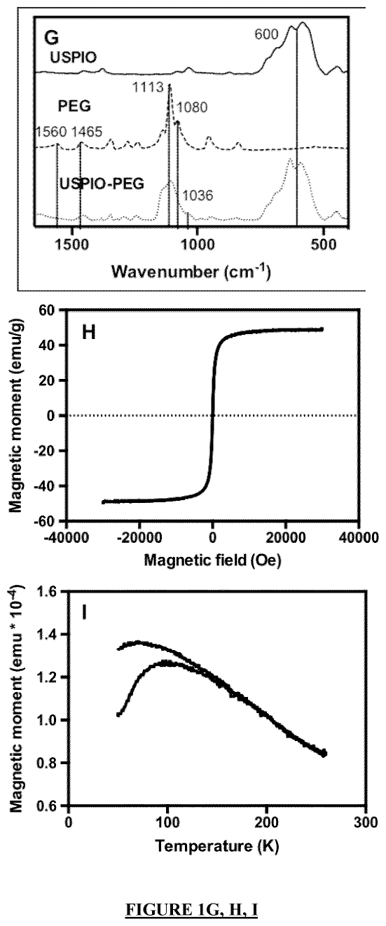

The USPIO-PEG NPs were characterized using several physicochemical methods (FIG. 1B to I). Transmission Electronic Microscopy (TEM) images at low magnification show well-dispersed NPs with spherical morphology and a narrow size distribution (FIGS. 1B and D). The high magnification TEM images show an organic layer of about 0.9 nm around the NPs (FIG. 1C) attributed to the PEG coating.

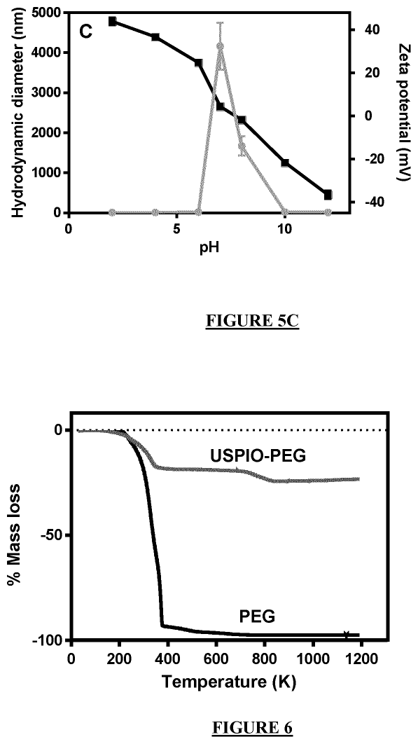

The negative zeta potential of the USPIO-PEG NPs at pH 7.4 clearly shows efficient binding of the PEG to the NP surface (FIG. 1D). This result was corroborated considering Fourier Transform Infra Red (FTIR) spectra of free PEG molecules and USPIO-PEG NPs (FIG. 1G). Free PO-PEG-COOH molecules (red curve, FIG. 1G) are mainly characterized by the PO asymmetric vibration bands ((vₐ(PO₃²⁻)) at around 1080 cm⁻¹, the characteristic band at 1113 cm⁻¹ due to the stretching vibration of aliphatic ether C—O and the carboxylate symmetric and asymmetric vibration bands at 1465 cm⁻¹ and 1560 cm⁻¹. . . a broadening of the band at 1113 cm⁻¹, a blue shift of the PO vibration band at 1036 cm⁻¹ and of the symmetric carboxylate band at 1455 cm⁻¹ whereas the asymmetric vibration band disappears, These results suggest that the complexation on NP surface could potentially occur either through the phosphonate or carboxylate groups. Nevertheless, we will show later that the P3UmPI protein will be covalently linked to nanoparticle surface using carbodiimide chemistry indicating that some COOH are at the outer surface of the NP. The number of PEG molecules per particle was determined by ThermoGravimetric Analysis (TGA) analysis (FIG. 6). An average of 180 PEG molecules per NP was determined. This corresponds to an average density of 0.7 PEG/nm². Hence the average distance between two PEG chains on the NP, D=(S_{NP}/Nb_{PEG})^{1/2}, with S being the surface area, is around 1.2 nm. Thus, considering the surface area of the phosphonate group (0.24 nm²) and the molecular polymer weight (M_{W}=2100 g.mol⁻¹), the corresponding Flory radius found equals to 3.4 nm.

The distance D is inferior to RF suggesting a brush conformation after PEG grafting onto the NP surface. After the coating process, the magnetization saturation (M_{S}=49 emu.g⁻¹, FIG. 1G) remained similar while the blocking temperature decreased (T_{b}=100K, FIG. 1I) compared to bare NPs (FIG. S1). This decrease of T_{b} is in accordance with efficient binding of PEG onto the NP surface. The PEG coating decreases the attractive magnetic dipole interaction in regard to steric and/or electrostatic repulsive interactions. FIG. 1F shows highly stable dispersion over time for up to 100 h in various media (pH 7.4 and 37° C.) including water, NaCl 0.9%, NaCl 0.9%/Glucose 5%.

### Example 2: Evaluation of the Coupling and Packaging Efficiency for ITE and/or the P3 UmPI Protein on the USPIO-PEG Nanoplatform

As described previously, our objective was to elaborate a hybrid nanoplatform using USPIO as core nanosubstrate for vectorization of ITE together with the fusion protein P₃UmPI, with the additional potential to use the inorganic core as MRI contrast agent in order to study biodistribution. For this, we first characterized the coupling of each molecule before optimizing the full nanosystem.

ITE is a hydrophobic molecule soluble only in DMSO and ethanol. We took advantage of the PEG brush conformation on the NP surface to trap the molecules between PEG chains using hydrophobic interactions. After NP incubation at room temperature for 2 hours with a ratio R_{ITE/NP}=600 followed by NP washing (Methods), no significant zeta potential and hydrodynamic size modification was observed (Table 1).

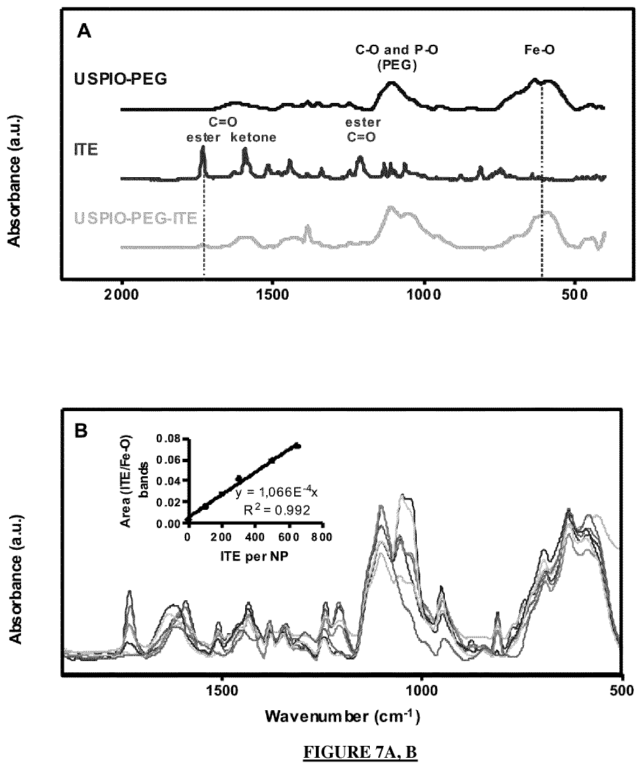

We first evaluated ITE loading by FTIR spectroscopy (FIG. 7) and an average of 250 ITE per NP was deduced. Seeking a more accurate method, we quantified ITE after drug release and water solubilization. Basic hydrolysis (NaOH 1M, 2 hours) of ITE induces the formation of 2-(1′H-indole-3′-carbonyl)-thiazole-4-carboxylic acid (carboxylate ITE) and methanol as shown on H NMR spectra. Carboxylate ITE is water-soluble and presents two characteristic UV bands at 279 nm and 388 nm, with an absorbance directly related to the concentration of carboxylate ITE (FIGS. 1J and K). The amount of ITE per NP was deduced after basic hydrolysis of USPIO-PEG-ITE (in the same conditions as free ITE), NP separation and titration of carboxylate ITE in the supernatant (Methods). An average number of 348±70 ITE per NP was deduced, corresponding to a yield of 58%. Considering the average number of 180 PEG molecules per NP, this indicates that an average of two ITE molecules are trapped between two PEG chains. Comparing the results deduced with FTIR spectroscopy, a rather good agreement is obtained between the two methods. Finally ITE drug release in NaCl 0.9%/glucose 5% media was also evaluated at various time points, using quantitative (UV spectroscopy) assay. We found that about 9% of the drug loaded is released after 5 hours and that a plateau is reached at 24 h that remains stable up to 72 h, the maximum duration of in vitro experiments in this work, (FIG. 10).

The protein P3UmPI, (38.7 kDa; −7.7 mV at pH =7.4), was expressed in E. Coli BL21pET16 DE3 (Methods). P3UmPI is a fusion protein composed of murine proinsulin (mPI) which is the primary autoantigen triggering autoimmune diabetes in NOD mice, according to You & Chatenoud (“Proinsulin: a unique autoantigen triggering autoimmune diabetes”; J. Clin. Invest.; 2006, 116(12); 3108-10).

In the fusion protein, mPI is preceded by tandem immunoglobulin-binding domains from streptococcal protein G to allow for binding of the fusion proteins to Abs. Between these domains and mPI, ubiquitin (Ub) is inserted upstream which enhances proteasomal degradation of proteins linked to its C terminus, as described in Qian et al. (“Fusion Proteins with COOH-terminal Uniquitin Are Stable and Maintain Dual Functionality in Vivo”; The Journal of Biological Chemistry; 2002; Vol. 277; No. 41; pp. 38818-38826).

The classic carbodiimide reaction was used to conjugate the protein to USPIO-PEG NPs. This reaction was performed at molar ratios R_{P3UmPI/NP}=8. Compared to UPSIO-PEG NPs, P3UmPI-incubated NPs doesn't show any drastic change of the hydrodynamic size and a more negative surface charge (Table 1). In addition the FTIR spectrum of USPIO-PEG-P3UmPI shows the characteristic amide bands I (1650 cm⁻¹), II (1550 cm⁻¹), and III (1300 cm⁻¹) of the free protein (black curve); according to the methodology detailed in Pelton & McLean (“Spectroscopic Methods for Analysis of Protein Secondary Structure”; Analytical Biochemistry; 2000; 277; 167-176).

TABLE 1
Hydrodynamic diameter (in number and volume), polydispersity and
Zeta potential of various batch of nanoparticles in water, pH = 7.

| | | pH = 7 | | | |
|---|---|---|---|---|---|
| | | | | USPIO- | USPIO- |
| | pH = 2 | USPIO- | USPIO- | PEG- | PEG-ITE- |
| | USPIO | PEG | PEG-ITE | P3UmPI | P3UmPI |
| Hydrodynamic | 9.4 | 12.6 | 12.4 | 11.7 | 11.6 |
| diameter in: | | | | | |
| number (nm) | | | | | |
| Hydrodynamic | 14.7 | 14.9 | 19.0 | 18.5 | 17.4 |
| diameter in: | | | | | |
| volume (nm) | | | | | |
| Polydispersity | 0.1 | 0.3 | 0.4 | 0.4 | 0.4 |
| index | | | | | |
| Zeta potential | 42.7 | −13.9 | −19.5 | −22.4 | −22 |
| (mV) | | | | | |

The o-phthalaldehyde (OPA) method was used for quantification of amino groups after chemical decomposition of the amide coupling on NPs. An average of 4.3±0.3 proteins per NP was found, corresponding to a yield of 50%.

To functionalize the USPIO-PEG nanoplatform with both ITE and the protein P₃UmPI, the NPs were first loaded with ITE and the protein was grafted in a second step. ITE saponification and OPA titration indicated an average of 345 ITE and 4.0±0.5 proteins per NP, corresponding to a similar loading as compared to the two independent nanoplatforms. Hence, different physicochemical techniques confirmed successful co-packaging of ITE and protein onto the USPIO-PEG nanoplatform.

### Example 3: Magnetic Behavior and MRI Relaxivities

To complete characterization of our nanoplatform, which involves iron oxide nanoparticles, magnetic and T₂ MRI contrast agent properties were evaluated. The different bare and loaded forms of USPIO show superparamagnetic behavior at room temperature with a magnetization at saturation around 50 emu.g⁻¹ and a blocking temperature around 100K.

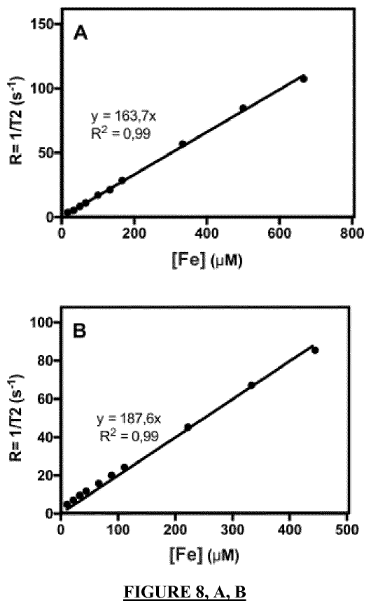

The relaxation time was measured on a 7 T MR scanner (FIG. 8). The r₂ relaxivity of USPIO-PEG and USPIO-PEG-ITE-P3UmPI, were 163.7 and 187.6 mM⁻¹·s⁻¹, respectively. The increased transverse relaxivity r2 of the loaded platform may be related to the increase in hydrodynamic size. These r₂ values are slightly higher than those of commercial MRI contrasts agents such as than Resovist (177 mM⁻¹·s⁻¹) and Endorem (160 mM⁻¹·s⁻¹), confirming the significant potential of these NPs as T₂-shortening contrast agents for contrast-enhanced MRI applications.

### Example 4: Studying Uptake and Toxicity of Nanoplatforms for Murine BMDCs

Prior to in vivo experiments, uptake and toxicity of the different nanoplatforms were assessed in vitro using murine BMDCs, professional antigen presenting cells (APCs) with a key role in immune responses and tolerance, as model.

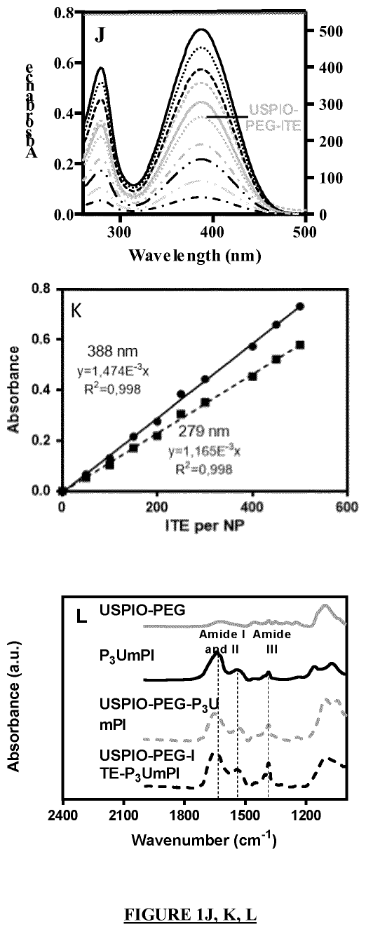

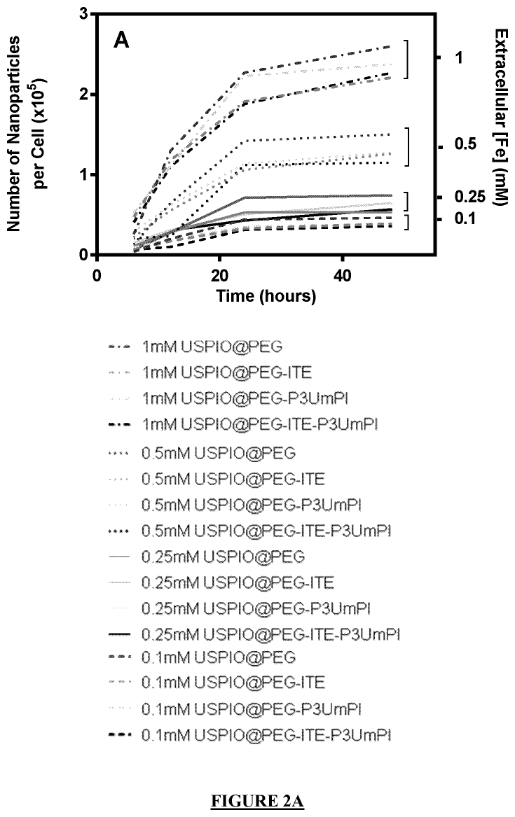

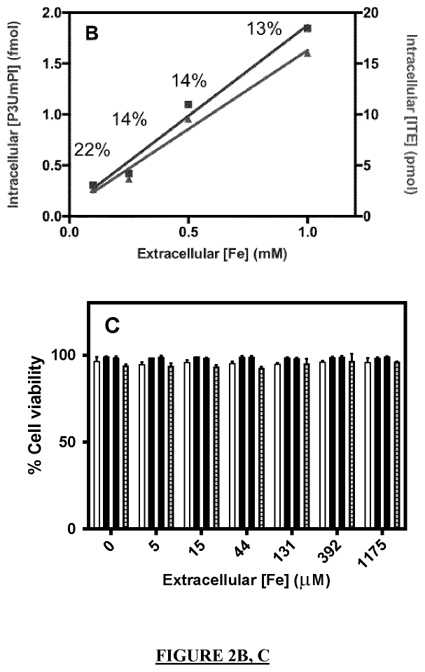

To quantify internalization of the various nanoplatforms by the cells, field-dependent magnetization curves were measured at 300 K using 14.000.000 cells. Taking into account the average NP size, the average number of NP per cell is deduced from the saturate magnetization (Methods). FIG. 2A shows that NP uptake increased with the extracellular NP concentration. Whatever the extracellular NP concentration used, cell saturation was observed after 24 h of NP incubation (FIG. 2A). Interestingly, the percentage of internalized particles remained constant for all studied concentrations, suggesting establishment of equilibrium of NP distribution between the extra and intracellular space within 24 h (FIG. 2B).

BMDC viability was assessed 24 h after NP incubation using a flow cytometric assay. FIG. 2C shows that no cytotoxicity is observed even at high NP extracellular concentration (1 mM iron, corresponding to 15% intracellular NP internalization).

### Example 5: Biological Validation of the Presence and Activity of ITE and P3 UmPI on USPIO-PEG

ITE is an aryl hydrocarbon receptor (AhR) ligand. This receptor forms a negative regulatory loop with its repressor, aryl hydrocarbon receptor repressor (AhRR), which is induced in response to AhR activation. ITE and USPIO-PEG-ITE were incubated in vitro with BMDCs at identical ITE extracellular concentration, and AhRR expression was measured by qPCR. The results, displayed in FIG. 2D, clearly show that AhRR expression is induced by both USPIO-PEG-ITE NPs and free ITE, with a possible advantage for the former. This difference could be related to better internalization of ITE nanocarriers compared to hydrophobic free ITE. This also shows that biologically active ITE is released from NPs.

The integrity of NP-loaded fusion protein was confirmed by western blot and immunostaining. As NPs are designed as theranostic devices to be used in the context of autoimmune diabetes, proinsulin functionality can be defined as its recognition by T and B lymphocytes, both implicated in the pathogenesis of this disease. P3UmPI was detected on USPIO-PEG-ITE-P3UmPI by gel electrophoresis followed by immunostaining with an antibody recognizing the insulin B chain (FIG. 2E). The Western blot demonstrates that full length proinsulin fusion protein is associated with NP.

### Example 6: MRI In Vivo Experiments

T1D development is invariably associated with changes in vascular permeability in pancreatic blood vessels. This increased microvascular permeability is observed already before high blood glucose diagnosis in prediabetic mice.

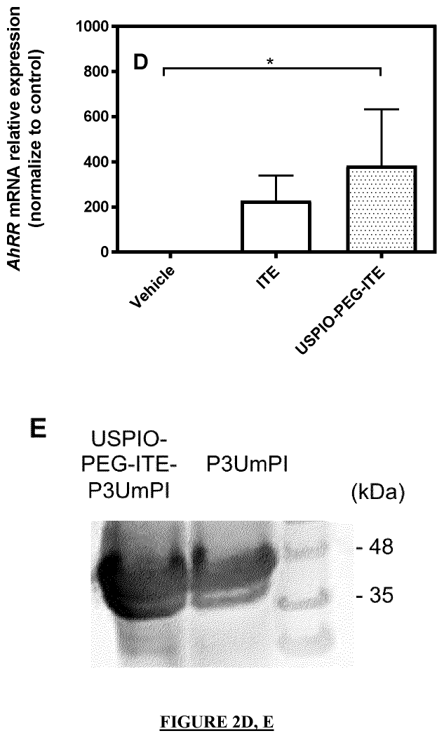

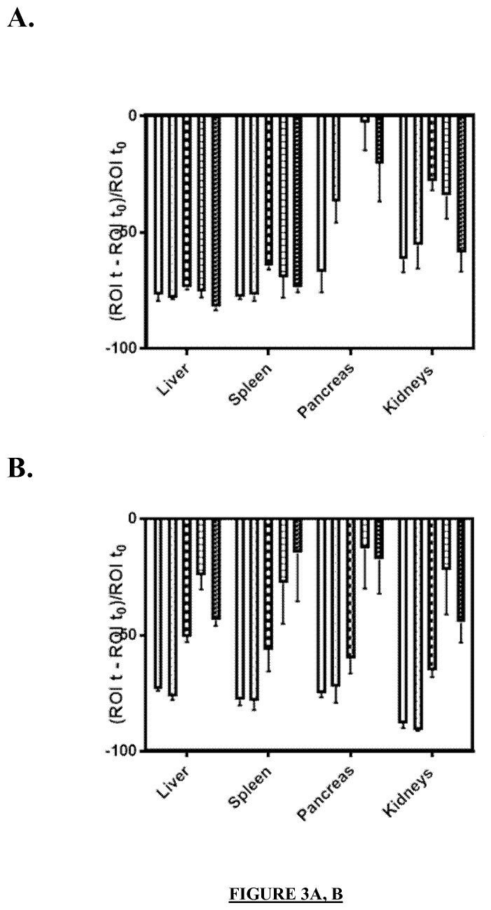

Since USPIO nanoparticles are a negative T2 contrast agent, local changes were used in T2 to evaluate the NP biodistribution in pre-diabetic NOD mice and control B6 mice using 7T-MRI. A 200 μmol iron/kg bolus was selected, corresponding to 1.3 nmol (50 μg) of P₃UmPI and 4.3 mmol of ITE. Liver, spleen, pancreas, and kidneys were scanned before injection and 30 min, 1 h, 5 h, 24 h and 72 h after NP injection. FIG. 3 shows MRI contrast variation for the various organs (FIG. 3A,B) for the two mice analyzed at different times after intravenous NP injection.

After injection, different NP biodistributions were observed in pre-diabetic NOD mice and non-diabetic controls. Shortly after injection (t=30 min) and 1 hour after, a large negative contrast enhancement was observed across organs rich in macrophages, i.e. liver, kidneys, spleen but also in pancreas. These results show that NPs are still circulating. Moreover, considering that contrast intensity is related to NP concentration, it can be observed that contrast in pancreas (t=1 h) is greater in NOD mice than in controls, presumably reflecting islet inflammation. Negative contrast is also observed in kidneys indicating NP elimination, once again stronger for NOD mice. Kidney filtration is a desirable pathway for NP clearance because potential health hazards resulting from long-term accumulation and decomposition of NPs in the body can be minimized. In order to escape from the vascular compartment, NPs have to pass through fenestrated endothelium (70-90 nm), glomerular basement membrane (GBM, meshwork structure with 2-8 nm pores), and epithelial filtration slits (4-11 nm) between the podocyte extensions, i.e. three layers of glomerular capillary wall. Considering the combined effects of each layer, it is expected that small NP with a hydrodynamic diameter (HD) can pass through the glomerular capillary wall easily, while larger ones would not be filtrated. However, surface functionalization (charge) and opsonization effects also contribute to this effect. Considering the hydrodynamic size of the USPIO-PEG-ITE-P3UmPI nanoplatform (≈12 nm) and its polydispersity index (0.5), it is expected that smaller NPs are first eliminated via kidneys.

MRI images suggest a greater renal clearance for NOD mice than for controls. This could be due to renal hypertrophy and structural alterations of the proximal straight tubules in prediabetes in NOD mice. Both 5 h and 24 h after injection, NP biodistribution differ greatly between pre-diabetic and control mice. In control mice, a slight decrease of MRI contrast was observed in liver, spleen and kidneys and no signal was detected in pancreas. NOD mice displayed a greater contrast decrease in liver, spleen and kidneys whereas the contrast signal increased in pancreas. The difference in the pancreatic MRI signal clearly showed that NPs accumulated preferentially in the pancreas of pre-diabetic NOD mice but not in control pancreas. Hence, this result indicates that this nanoplatform can be used as a potential MRI contrast agent, especially for early diagnosis of T1D.

Surprisingly, 72 h after injection, MRI negative contrast accumulation was enhanced particularly in liver, pancreas and kidneys in both strains though more so in NOD mice, suggesting recirculation of NPs between 24 and 72 hrs after injection.

### Example 7: Quantification of NP Uptake in Various Organs Using Magnetometry

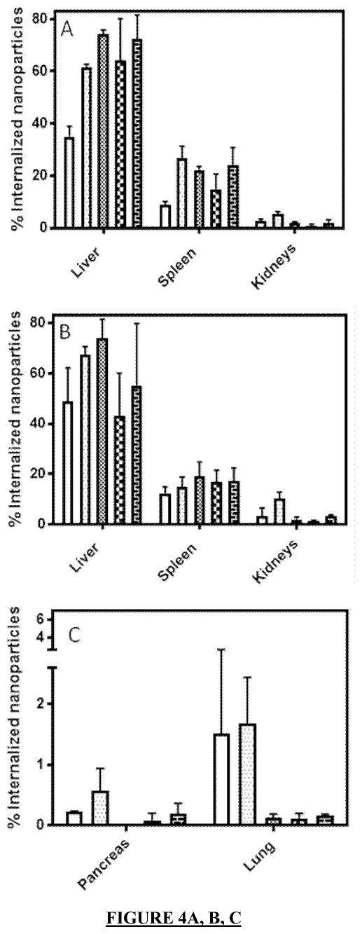

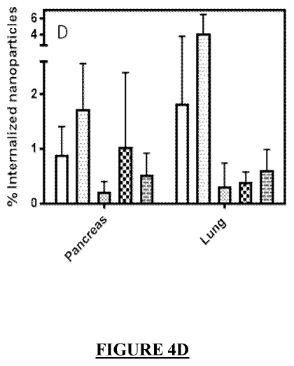

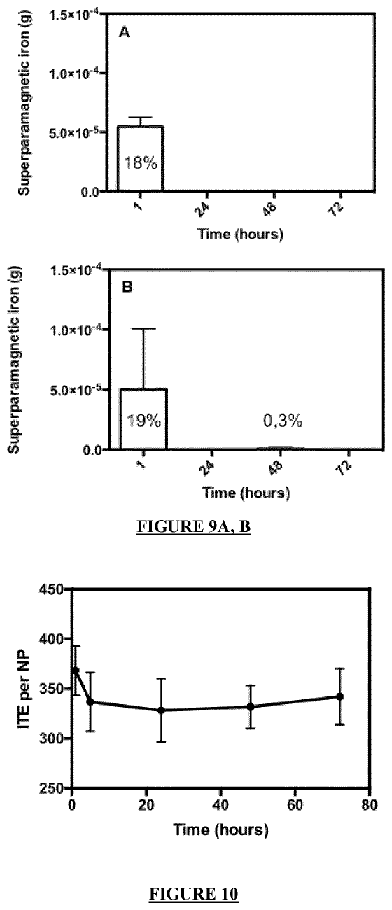

In order to confirm the results obtained by imaging, quantitative evaluations were performed using VSM measurements on blood (FIG. 9) and on various organs of interest (liver, kidney, spleen, lung and pancreas) (FIG. 4A to D). Prussian blue staining on histological sections was also carried out (FIG. 4) for qualitative assessment. Magnetometry quantification (FIG. 4A to D) confirmed MRI results and demonstrated much stronger accumulation in the pancreas of NOD mice at every time point but especially 5 h after injection, (Table 2).

TABLE 2
estimation of drug dose released in pancreas at various
time point after intraveinous (iv) injection, considering
no drug release before pancreas targeting.

| | ITE (mmol) | | P3UmPI (μmol) | |
|---|---|---|---|---|
| | B6 | NOD | B6 | NOD |
| 1 H | 0.39 | 1.54 | 4.53 | 17.87 |
| 5 H | 0.99 | 2.98 | 11.42 | 34.52 |
| 24 H | 0 | 0.37 | 0 | 4.33 |
| 48 H | 0.15 | 1.78 | 1.73 | 20.67 |
| 72 H | 0.33 | 0.91 | 3.8 | 10.6 |

Elimination in NOD liver seemed to be faster compared to B6 controls and kidney elimination, especially at 5 h, was also stronger in NOD compared to controls (*: p=0.0143). Low magnetic field (LMF) magnetization analysis was then performed on livers and kidneys 5 h after injection. This LMF analysis allows to estimate NP size distribution.

In B6 mice, kidneys present a lower initial magnetic size compared to the size distribution in liver. This suggests that kidneys eliminate smaller particles related to size distribution. Interestingly, there is no magnetic size difference in the kidney signal of NOD mice compared to the signal for B6 and NOD mouse livers. This is consistent with the notion that structural alterations affecting the kidneys of NOD mice allow to eliminate bigger NPs. These data, consistent with MRI and magnetometry observations, are also in accordance with histological examination of kidney, revealing that the structure of NOD kidneys is altered compared to kidneys of B6 mice.

Hence, magnetic analysis confirmed a re-accumulation of NPs both in NOD and B6 pancreas at 24 h and in livers at 72 h after injection, consistent with recirculation of NPs. It is clearly observed that NPs are eliminated by kidneys, accumulate in pancreas for NOD mice and 48 h after intravenous injection that NP re-circulate in the organism, corroborating MRI analysis.

### Example 8: Histological Studies

While suitable for examining larger structures such as pancreas and liver, MRI studies don't allow for evaluating NP biodistribution in pancreatic lymph nodes (PLN). Considering that diabetogenic T cell responses are initiated in PLN, PLN appear as a major target for tolerogenic treatment.

In order to evaluate the ability of the nanoplatform to reach pancreas-draining lymph nodes, histological studies with Perl's staining were performed on lung, liver, spleen, pancreas, PLN and kidneys removed at different time points after injection. Histological images 5 hours after injection confirm NP accumulation in liver, spleen, pancreas and also PLN.

### Example 9: In Vivo Studies on Prediabetic and Diabetic Mice Treated With Functionalized Nanoparticles According to the Invention

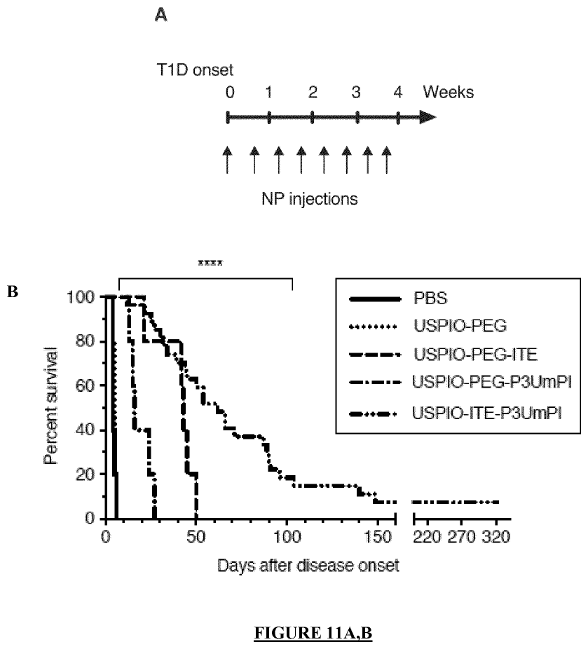

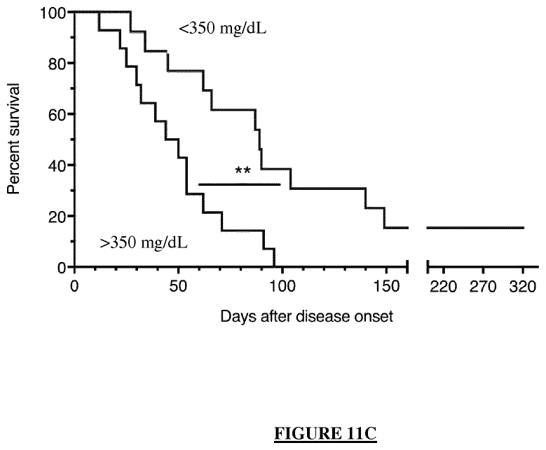

We have evaluated the effect on glycemia of treating pre-diabetic NOD by injecting NPs twice weekly during four weeks (FIG. 11A). Relative to vehicle or “naked” (PEG-coated only) NPs, particles loaded with P3UmPI or ITE only delayed terminal hyperglycemia (≥600 mg/dL) by up to 27 or 45 days, respectively. In contrast, NPs loaded with both ITE and P3UmPI delayed terminal hyperglycemia up to 150 days, and cured 2 mice indefinitely (FIG. 11B).

Mice displaying glycemia below 350 mg/dL at start of treatment responded better, with 50% survival at 80 days vs. 10% for mice with initial glycemia above 350 mg/dL (FIG. 11C).

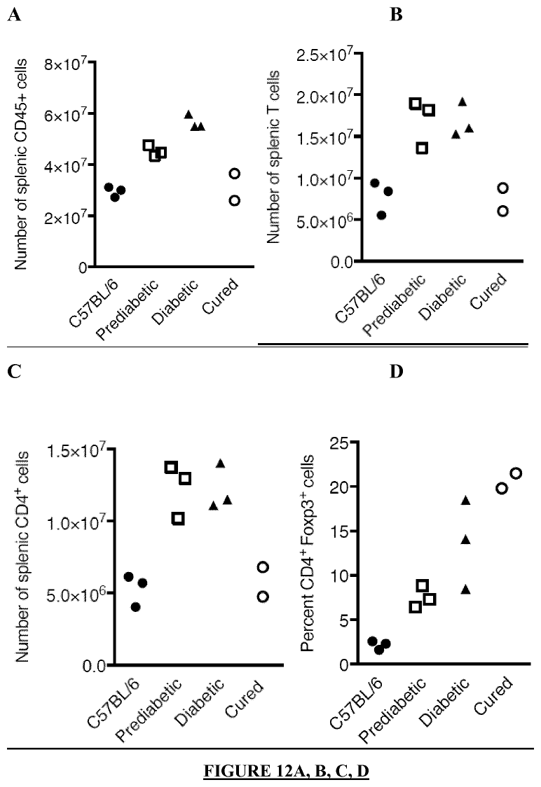

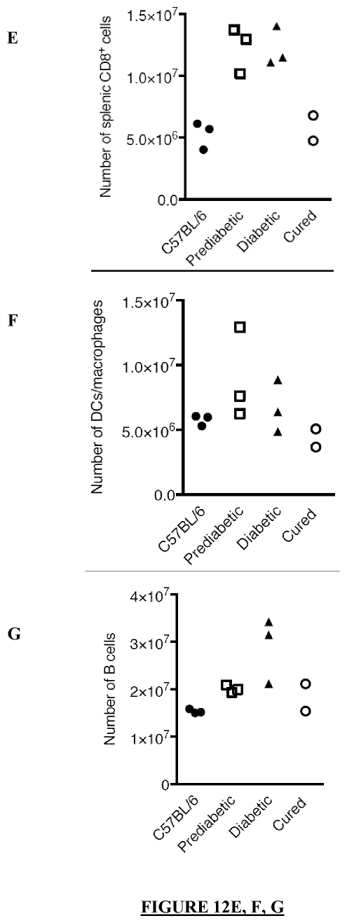

Finally we analyzed splenic immune cells in the 2 mice cured by complete NP treatment. Interestingly, these mice resembled non-autoimmune C57BL/6 but not untreated pre-diabetic or diabetic NOD mice with respect to the numbers of cells of the innate and adaptive immune system (FIG. 12), however they exhibited a strong increase in the percentage of Foxp3+ regulatory T cells in both spleen (similar results were obtained for pancreatic lymph node cells). Accordingly histological insulitis showed strongly attenuated insulitis.

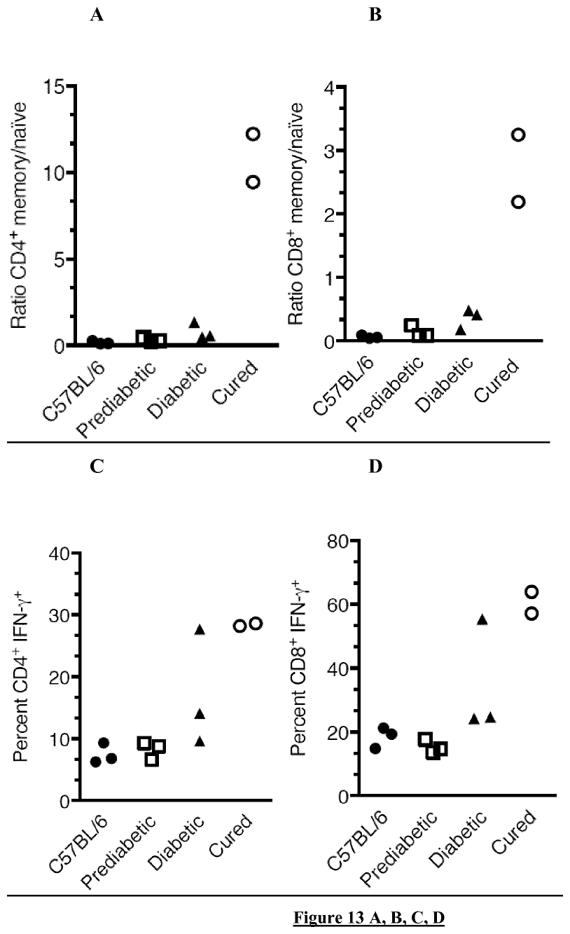

Another striking feature distinguishing cured mice both from non-autoimmune C57BL/6 and prediabetic or diabetic NOD mice was the very high ratio of memory to naïve CD4+ and CD8+ T cells associated with high proportions of IFN-γ-producing T cells FIG. 13 A-D), both in spleen and PLN. Therefore IFN-γ producing memory/activated T cells are likely to play a role in the curative effect of complete NPs.

1. A method for treating type-I diabetes in a subject in need thereof, comprising administering to the subject a biocompatible tolerogenic nanoparticle comprising at least:
(i) a ligand which can bind to an aryl hydrocarbon receptor (AHR) transcription factor; and
(ii) a diabetes autoantigen selected from: insulin, preproinsulin, proinsulin, or an immunologically active fragment thereof.

2. A method for the in vivo diagnosis of type-I diabetes in a subject in need thereof comprising administering to the subject a biocompatible tolerogenic nanoparticle comprising at least:
(i) a ligand which can bind to an aryl hydrocarbon receptor (AHR) transcription factor; and
(ii) a diabetes autoantigen selected from: insulin, preproinsulin, proinsulin, or an immunologically active fragment thereof.

3. A biocompatible tolerogenic nanoparticle comprising at least:
(i) a ligand which can bind to an aryl hydrocarbon receptor (AHR) transcription factor; and
(ii) a diabetes autoantigen selected from: insulin, preproinsulin, proinsulin, or an immunologically active fragment thereof;
wherein the said nanoparticle is a magnetic nanoparticle.

4. A biocompatible tolerogenic nanoparticle comprising at least:
(i) a ligand which can bind to an aryl hydrocarbon receptor (AHR) transcription factor;
(ii) a diabetes autoantigen selected from: insulin, preproinsulin, proinsulin, or an immunologically active fragment thereof;
wherein the nanoparticle has an average size of less than about 50 nm.

5. The biocompatible tolerogenic nanoparticle according to claim 3; wherein the said nanoparticle has an average size of less than about 50 nm.

6. The biocompatible tolerogenic nanoparticle according to claim 3; wherein the diabetes autoantigen is in the form of a fusion protein characterised in that the fusion protein comprises:
an IgG binding moiety, and
as a cargo moiety, a polypeptide comprising a sequence selected from the group consisting of insulin, preproinsulin, proinsulin, and an immunologically active fragment thereof.

7. The biocompatible tolerogenic nanoparticle according to claim 4; wherein the nanoparticle is a magnetic nanoparticle.

8. The biocompatible tolerogenic nanoparticle according to claim 3; wherein the nanoparticle is an iron oxide nanoparticle.

9. The biocompatible tolerogenic nanoparticle according to claim 3; wherein the ligand which can bind to an aryl hydrocarbon receptor (AHR) transcription factor is 2-(1′H-indole-3′-carbonyl)-thiazole-4-carboxylic acid methyl ester (ITE).

10. The biocompatible tolerogenic nanoparticle according to claim 3; wherein the diabetes autoantigen is a polypeptide comprising a sequence selected from the group consisting of proinsulin and an immunologically active fragment thereof.

11. The biocompatible tolerogenic nanoparticle according to claim 3; wherein the nanoparticle has an average size of less than about 20 nm.

12. The biocompatible tolerogenic nanoparticle according to claim 3; wherein the nanoparticle is functionalized with phosphonate polyethylene glycol (PEG) molecules.

13. The biocompatible tolerogenic nanoparticle according to claim 12; wherein the average density of PEG molecules at the surface of the nanoparticle is from 0.5 to 2 PEG per nm².

14. The biocompatible tolerogenic nanoparticle according to claim 3; wherein the nanoparticle is linked to a ligand which can bind to an AHR transcription factor with an average density of ligand at the surface of the nanoparticle from 0.5 to 4 ligands per nm².

15. A composition comprising a contrast agent, in combination with the biocompatible tolerogenic nanoparticle of claim 3.

16. A kit comprising:
a first container containing the biocompatible tolerogenic nanoparticle of claim 3; and
a second container containing a contrast agent.

17. A method for preparing a contrast composition, comprising a step of bringing into contact a contrast agent with the biocompatible tolerogenic nanoparticle of claim 3.

18. A method for preparing a contrast composition, comprising a step of bringing into contact the biocompatible tolerogenic nanoparticle of claim 4 with (i) a ligand which can bind to an aryl hydrocarbon receptor (AHR) transcription factor, and (ii) a diabetes autoantigen selected from: insulin, preproinsulin, proinsulin, or an immunologically active fragment thereof.

19. The biocompatible tolerogenic nanoparticle according to claim 14, wherein the ligand is 2-(1′H-indole-3′-carbonyl)-thiazole-4-carboxylic acid methylester (ITE).
